(19) United States
(12) Patent Application Publication
(10) Pub. No.: US 2024/0248111 A1
(43) Pub. Date: Jul. 25, 2024

(54) DIAGNOSTIC TEST DEVICE WITH INTERNAL CYLINDERS AND PLUNGER

(71) Applicant: BECTON, DICKINSON AND COMPANY, Franklin Lakes, NJ (US)

(72) Inventors: Patrick Walter Truitt, New Freedom, PA (US); Alyssa Nicolaisen Shedlosky, Reisterstown, MD (US); Ammon D. Lentz, York, PA (US); Patrick Downie, Apex, NC (US); John Scott Wheeler, Bowling Green, OH (US); Benjamin Zoeckler, Bethel Park, PA (US)

(21) Appl. No.: 18/587,708

(22) Filed: Feb. 26, 2024

Related U.S. Application Data
(63) Continuation of application No. PCT/US2022/075918, filed on Sep. 2, 2022.
(60) Provisional application No. 63/241,033, filed on Sep. 6, 2021.

(51) Int. Cl. G01N 35/10; B01L 3/00

(52) U.S. Cl. CPC G01N 35/1079; B01L 3/5029; B01L 2200/0689; B01L 2300/042

(57) ABSTRACT

A diagnostic test device to perform a test on a biological or environmental sample is provided. In one aspect, the diagnostic test device includes a sample preparation reservoir; a dispensing mechanism that includes a sealing member and a piercing member; and at least one diagnostic test reservoir separated from the sample preparation reservoir by a seal. A predetermined volume of fluid is defined between the sealing member, the piercing member, sides of at least one chamber defined by the sample preparation reservoir, and the seal when the sealing member engages the sides of the at least one chamber. The piercing member disrupts the at least one seal to dispense the predetermined volume of fluid to the at least one diagnostic test reservoir. The sealing member is configured to directly contact the sides of the at least one chamber as the seal is disrupted.

### CROSS-REFERENCE TO RELATED APPLICATIONS

This application is a continuation of PCT Application No. PCT/US2022/075918, filed Sep. 2, 2022, which claims priority to U.S. Provisional Application No. 63/241,033, filed Sep. 6, 2021, each of which is hereby incorporated by reference in its entirety.

### BACKGROUND

### Technological Field

The present disclosure relates to devices, systems, and methods for performing a test on a sample-containing fluid. In particular, devices, systems, and methods of the present disclosure relate to a device configured to receive a sample for performing diagnostic tests or analysis of biological, chemical, or environmental samples to determine the presence and/or quantity of one or more analytes of interest in the sample. The analyte may be detected, for example, using DNA or RNA amplification when the device is received in a test system. Devices according to the present disclosure can include consumable devices for diagnostic testing, for example a disposable container that receives a sample, contains the sample before and during a diagnostic test, and is discarded to waste after the diagnostic test is complete.

### Description of the Related Technology

The amplification of nucleic acids is important in many fields, including medical, biomedical, environmental, veterinary and food safety testing. Example methods of nucleic acid amplification include polymerase chain reaction (PCR) amplification and isothermal amplification.

Nucleic acid amplification can generate a large number of copies of a target genetic sequence in a test solution. Specific markers can be designed to link to the target sequences as part of a test assay. Once bound, the markers can provide a detectable signal, for example an optical signal, from the test solution. Changes in an optical signal can include changes in the color, opacity, bioluminescence, and/or fluorescence of the test solution. In the case of a fluorescence marker beacon, each marker molecule may be configured with a florescence quencher in close proximity to a fluorescence atom or arrangement of atoms. This marker molecule can be configured such that when selectively bound to a target nucleic acid sequence, the quencher and fluorophore are separated and a fluorescence signal can then be detected by the action of the fluorophore. In this arrangement, the florescence intensity of the target solution is indicative of the relative amount of target genetic material in the test solution. This signal can then be used to form the basis of a diagnostic test to determine the presence or absence and the relative quantity of the target material, or analyte of interest, in the sample under test.

Two or more markers may be included in a single test well which each may provide optical output based on bonding to different target nucleic acid sequences. Different sensors, or a sensor with two or more selective outputs can be used in conjunction with these two or more markers. For example, in a two-channel system, two different fluorophores may be used that can be detected by two different fluorescence sensors configured to detect emissions in the respective frequency ranges of each fluorophore. Thus, the two channels may be discriminated.

Such an approach can be used to provide a control channel. In an example control channel, test assay chemistry is configured such that the control target, for example a synthetic nucleic acid sequence, should always be present if the test process is run correctly. The output of the control channel may be used to confirm that a test process has been run correctly by the system and/or to confirm the validity of test results obtained by other channels measured by the system. This approach can be applied to a test of more than one target sequence within a single test well.

Multiple test wells may be used. Each well may run different amplification chemistries and/or a different set of target markers. Control channels, as discussed above, may be operated in one or more wells.

In certain approaches, a test sample is not sealed from the environment during the process of sample preparation and transfer into test containers within a test instrument. This exposure of the sample can present infection risks to users and others, and may also contaminate the test instrument and test area, resulting in incorrect diagnostic results in subsequent tests. Further, such exposure may risk contaminating the test sample itself.

### SUMMARY OF CERTAIN INVENTIVE ASPECTS

The devices, systems, and methods of the present disclosure each have several innovative aspects, no single one of which is solely responsible for its desirable attributes. Without limiting the scope of the present disclosure, its more prominent features will now be discussed briefly.

In one embodiment, a device is provided. The device includes a sample preparation reservoir that can receive a sample at a first end and comprising an interior surface defining sides of at least one chamber at a second end. The device includes at least one diagnostic test reservoir. The device includes at least one seal disposed between the sample preparation reservoir and the at least one diagnostic test reservoir. The device includes a dispensing mechanism that can be inserted into the first end of the sample preparation reservoir and translated toward the second end of the sample preparation reservoir, the dispensing mechanism including a piercing member and a sealing member. The sealing member can engage the sides of the at least one chamber as the dispensing mechanism translates toward the second end of the sample preparation reservoir, to define a predetermined volume of fluid defined between the sealing member, the piercing member, the sides of the at least one chamber at the second end of the sample preparation reservoir, and the at least one seal when the sealing member engages the sides of the at least one chamber. The piercing member can pierce the at least one seal after the predetermined volume is defined, and the sealing member and the piercing member can dispense the defined volume from the sample preparation reservoir to the at least one diagnostic test reservoir after the seal is pierced.

The sealing member can be configured to directly contact the interior surface defining the sides of the at least one chamber as the dispensing mechanism translates toward the second end of the sample preparation reservoir.

In some embodiments, the piercing member does not move relative to the sealing member as the defined volume is dispensed to the at least one diagnostic test reservoir.

The piercing member may include at least one spiked rod and the sealing member may include at least one gasket encircling the at least one spiked rod.

For some embodiments, a single action of translating the dispensing mechanism toward the second end of the sample preparation reservoir can (a) define the predetermined volume of fluid between the sealing member, the piercing member, the sides of the at least one chamber, and the at least one seal; (b) pierce the at least one seal; and (c) dispense the defined volume into the at least one diagnostic test reservoir.

The sample preparation reservoir, the at least one diagnostic test reservoir, and the at least one seal can be connected to form a joined structure.

The interior surface at the second end of the sample preparation reservoir can define at least one cylindrical chamber.

The interior surface at the second end of the sample preparation reservoir can define two cylindrical chambers.

The device can include a notch in a portion of the interior surface between the two cylindrical chambers, where the predetermined volume is defined, at least in part, by a depth of the notch.

The sealing member can be configured to be in direct contact with a lower interior surface of the at least one chamber when the defined volume has been dispensed from the sample preparation reservoir to the at least one diagnostic test reservoir.

The interior surface at the second end of the sample preparation reservoir can define two cylindrical chambers, each of the two cylindrical chambers configured to dispense the predetermined volume of fluid. The piercing member can include two spiked rods. The sealing member can include a gasket encircling each of the two spiked rods. The device can include a test container including two diagnostic test reservoirs, each diagnostic test reservoir configured to receive the predetermined volume of fluid from one of the two cylindrical chambers.

The interior surface at the second end of the sample preparation reservoir can define four chambers, and the device can include four diagnostic test reservoirs.

The sealing member can include an elastomeric material.

The piercing member can include one or more spikes. Each of the one or more spikes can include a cross-shaped cross-section including concave surfaces and a chamfered surface.

The sample preparation reservoir can include a sample preparation fluid.

The at least one seal may include a first seal configured to seal the second end of the sample preparation reservoir and a second seal configured to seal the diagnostic test reservoir.

The at least one seal may include a foil.

The sample preparation reservoir can be configured to receive a swab that includes the sample.

The second end of the sample preparation reservoir can include a lip configured to be joined to the diagnostic test reservoir.

The sample preparation reservoir can be configured to contain a volume of fluid ranging from 1-3 mL and the predetermined volume ranges between 10 μL and 1 mL.

The sample preparation reservoir can be configured to contain a fluid volume that is 1 to 300 times greater than the predetermined volume.

The sample preparation reservoir can be configured to contain a fluid volume of 1-3 mL and the predetermined volume is about 100 μL.

The dispensing mechanism can include a cap configured to engage the first end of the sample preparation reservoir. The cap can be configured to rotate relative to the piercing member. The first end of the sample preparation reservoir can include threads configured to engage threads of the cap. The cap can be configured to lock to the first end of the sample preparation reservoir, preventing substantial motion of the cap relative to sample preparation reservoir. The cap can include a plug seal configured to engage a top end of the sample preparation reservoir. The plug seal can be configured to block fluid flow when engaged to the top end of the sample preparation reservoir.

In another embodiment, a diagnostic test apparatus configured to receive the device is provided.

In still another embodiment, a method of performing a diagnostic testing using a diagnostic test device is provided. The diagnostic test device can include a sample preparation reservoir and at least one diagnostic test reservoir. The sample preparation reservoir may include a first end and an interior surface defining sides of at least one chamber at a second end. The method may include introducing a sample to a fluid within the sample preparation reservoir at the first end of the sample preparation reservoir. The method can include dispensing a predetermined volume of the fluid from the sample preparation reservoir to the at least one diagnostic test reservoir. Dispensing the predetermined volume of the fluid can include inserting a dispensing mechanism into the first end of the sample preparation reservoir; and translating the dispensing mechanism toward the second end of the sample preparation reservoir. Dispensing the predetermined volume of the fluid can include engaging a scaling member of the dispensing mechanism with the sides of the at least one chamber to define the predetermined volume between the sealing member, a piercing member of the dispensing mechanism, the interior surface defining sides of the at least one chamber, and at least one seal between the sample preparation reservoir and the at least one diagnostic test reservoir. Dispensing the predetermined volume of the fluid can include piercing the at least one seal between the sample preparation reservoir and the at least one diagnostic test reservoir with the piercing member of the dispensing mechanism. The method can include performing an amplification reaction in the at least one diagnostic test reservoir. The method can include detecting the presence or absence of an analyte of interest in the at least one diagnostic test reservoir.

Engaging the sealing member and defining the predetermined volume may occur simultaneously.

The interior surface at the second end of the sample preparation reservoir may define two cylindrical chambers, each of the two cylindrical chambers configured to dispense the predetermined volume of fluid. The piercing member can include two spiked rods. The sealing member can include a gasket encircling each of the two spiked rods. The diagnostic test device can include two diagnostic test reservoirs. The predetermined volume of fluid can be dispensed from each of the two cylindrical chambers to one of the diagnostic test reservoirs.

The interior surface at the second end of the sample preparation reservoir may define four cylindrical chambers. The device can include four diagnostic test reservoirs.

The sealing member may be in direct contact with a lower interior surface of the at least one chamber after the predetermined volume of the fluid is dispensed from the sample preparation reservoir to the at least one diagnostic test reservoir.

Translating the dispensing mechanism may cease by locking the dispensing mechanism in place relative to the sample preparation reservoir.

Locking may include engaging a locking thread of the diagnostic test device with a locking tab of a cap of the dispensing mechanism. Locking may further include engaging a blocking flange of the diagnostic test device with an overtravel tab of the cap of the dispensing mechanism.

The predetermined volume of fluid may begin to dispense to the at least one diagnostic test reservoir before the at least one seal is fully pierced.

The method may include rehydrating a lyophilized reagent within the diagnostic test reservoir with the volume of fluid. The lyophilized reagent may include nucleic acid amplification primers. The lyophilized reagent may include a nucleic amplification detection probe.

Performing the amplification reaction in the at least one diagnostic test reservoir may include applying heat to the at least one diagnostic test reservoir.

Detecting the presence or absence of an analyte may include measuring an optical signal from the at least one diagnostic test reservoir. Measuring the optical signal may include measuring a fluorescence from the at least one diagnostic test reservoir.

The method may further include engaging the diagnostic test device with a diagnostic test apparatus configured to perform the amplification reaction and detect the presence or absence of the analyte of interest. Engaging the diagnostic test device may include heating the sample preparation reservoir.

### BRIEF DESCRIPTION OF THE DRAWINGS

The above-mentioned aspects, as well as other features, aspects, and advantages of embodiments of the present disclosure will now be described in connection with various implementations, with reference to the accompanying drawings. The illustrated implementations are merely examples and are not intended to be limiting. Throughout the drawings, similar symbols typically identify similar components, unless context dictates otherwise.

FIG. 1 illustrates an exploded view of components of an example diagnostic test device according to the present disclosure.

FIG. 2 illustrates the example diagnostic test device of FIG. 1 with a dispense cap.

FIG. 3 illustrates the example diagnostic test device of FIG. 1 with a transportation cap.

FIG. 4A illustrates a view of a cartridge body of the embodiment depicted in FIG. 1.

FIG. 4B illustrates a view of cylindrical chambers of the embodiment depicted in FIG. 4A.

FIG. 4C illustrates a cross-sectional view of the cylindrical chambers of the embodiment depicted in FIG. 4A.

FIGS. 4D and 4E illustrate cross-sectional views of the cartridge body of the embodiment depicted in FIG. 4A.

FIG. 4F illustrates a side view of the cartridge body of the embodiment depicted in FIG. 4A.

FIG. 4G illustrates a top-down view of the cartridge body of the embodiment depicted in FIG. 4A.

FIG. 4H illustrates an enlarged view of the test container of FIG. 1.

FIG. 5A illustrates the dispense cap of FIG. 1.

FIG. 5B illustrates a cross-sectional view of another dispense cap according to the present disclosure.

FIG. 5C illustrates a cross-sectional view of the dispense cap of FIG. 5B engaged with the cartridge body of the embodiment of FIG. 1.

FIG. 5D illustrates the interaction of a locking tab of the dispense cap of FIG. 1 with a locking thread of the cartridge body of FIG. 1.

FIG. 5E illustrates the interaction of an overtravel tab of the dispense cap of FIG. 1 with a blocking flange of the cartridge body of FIG. 1.

FIG. 6A illustrates a side view of the dispensing mechanism of FIG. 1.

FIG. 6B illustrates an angled bottom view of the dispensing mechanism of FIG. 1.

FIGS. 6C and 6D illustrate an alternative embodiment of a sealing member according to the present disclosure.

FIGS. 6E and 6F illustrate top and bottom views, respectively, of the alternative embodiment of a sealing member depicted in FIGS. 6C and 6D.

FIG. 7A illustrates a cross-sectional view of the embodiment of FIG. 1, where the dispensing mechanism has been inserted into the sample preparation reservoir.

FIG. 7B illustrates a cross-sectional view of the embodiment of FIG. 1, where the dispensing mechanism has pierced the seal.

FIG. 7C illustrates a cross-sectional view of the embodiment of FIG. 1, where the sealing member has engaged the walls of the cylindrical chambers but has not pierced the seal.

FIGS. 7D and 7E illustrate a cross-sectional view of the embodiment of FIG. 1, where the dispensing mechanism has been fully inserted, and a predetermined amount of fluid has been dispersed to the diagnostic test reservoir. FIG. 7D is a view that shows the piercing members more closely than FIG. 7E, which shows the entire diagnostic test device.

FIG. 8 illustrates an example method for performing a diagnostic test using a diagnostic test device in accordance with the present disclosure.

FIG. 9 illustrates the diagnostic test device of FIG. 1 received in a portion of a diagnostic test apparatus.

FIGS. 10-61 illustrate views of an example diagnostic test device according to the present disclosure.

### DETAILED DESCRIPTION

Embodiments of the present disclosure provide devices, systems, and methods that consistently transfer a predetermined volume of a solution, such as a fluid sample, from one portion of a diagnostic test device to another portion of the device, while avoiding contamination of the solution and the external environment. The fluid sample can include a test sample in a buffer solution. In some cases, the fluid sample is amplification-ready when it is transferred from the first portion to the second portion of the device. The first portion of the diagnostic test device can include a sample preparation reservoir and the second portion of the diagnostic test device can include one or more test containers. For example, a predetermined amount of the fluid sample can be transferred from a sample processing reservoir to one or more test containers that include pre-stored amplification reagents. The sample processing device can include dual internal cylinders, and the predetermined amount of the fluid sample can be dispensed through the dual internal cylinders to two test containers using a plunger. Prior to transfer of the fluid sample, the test containers are sealed to the external environment and the sample preparation reservoir, and are thus protected from contaminants. After the transfer of the fluid sample, the test containers remain sealed to the external environment. Advantageously, the external environment is not exposed to the fluid sample, which can include hazardous components.

Diagnostic test devices of the present disclosure can dispense a predetermined amount of the fluid sample at the same time a sample-receiving end of the sample preparation reservoir is sealed. For example, the action of twisting a cap engaged to the sample-receiving end of the sample preparation reservoir also dispenses the fluid sample from the sample preparation reservoir to the test containers. Upon dispensing the fluid sample, the cap can lock, preventing access to the sample preparation reservoir and test containers, protecting them from contamination. Additional fluid flow between the sample preparation reservoir and the test containers is also prevented. The mechanism for dispensing the fluid sample while simultaneously sealing the diagnostic test device is uncomplicated, involving the movement of a single component within the sample processing reservoir. In particular, the dispensing mechanism includes a plunger configured to directly contact interior surfaces of the sample processing reservoir as the plunger translates within the sample preparation reservoir and a piercing end of the plunger pierces one or more seals separating the sample preparation reservoir and the test containers. Once dispensed, the fluid sample within the test containers may be assayed, using an amplification reaction for example, to determine the presence or absence of a target analyte. Advantageously, diagnostic test devices of the present disclosure can reliably dispense a precise volume of fluid sample from a single sample preparation reservoir into two or more test containers storing different reagents, allowing multiplex testing of a single sample.

Embodiments of the present disclosure provide devices, systems, and methods capable of preparing a test sample and subsequently testing the test sample, for example by amplification in conjunction with fluorescent markers. An embodiment includes a diagnostic test assembly (also referred to herein as a “cartridge”) for use with a diagnostic test instrument to perform a diagnostic test on a biological or environmental sample. Such a cartridge may be used with a diagnostic test apparatus (also referred to herein as an “instrument”). As described herein, the cartridge is easy for a user to operate without requiring the facilities of a general test laboratory.

Throughout the following description, various embodiments will be described with reference to an example implementation of a rapid, nucleic acid-based diagnostic system that may test for a variety of diseases. As illustrative examples, the system may test for sexually transmitted infections (STIs), such as gonorrhea and chlamydia, and respiratory tract infections (RTIs), such as influenza A or B. The example system is targeted to the Point of Care (POC) market where ease of use, simplicity, CLIA waivability and rapid turnaround time (TAT) of results are considerations. It will be understood, however, that any of the devices, systems, and methods described herein may be applied to any other medical, forensic, or other application.

The present disclosure relates to devices, systems, and method capable of carrying out amplification, such as isothermal amplification, of nucleic acids in a sample. Unless specifically made clear to the contrary, where the term amplification is used herein, any variant of amplification, including but not limited to isothermal amplification and PCR amplification (including real-time and quantitative PCR), is intended to be encompassed. It will be understood that devices, systems, and methods of the present disclosure are not limited to amplification of nucleic acids, and can test a sample for the presence or absence of any target of interest. It will also be understood that devices, systems, and methods of the present disclosure are not limited to processing or preparing a sample before the sample is tested for the presence or absence of a target on interest.

### Example Diagnostic Test Device

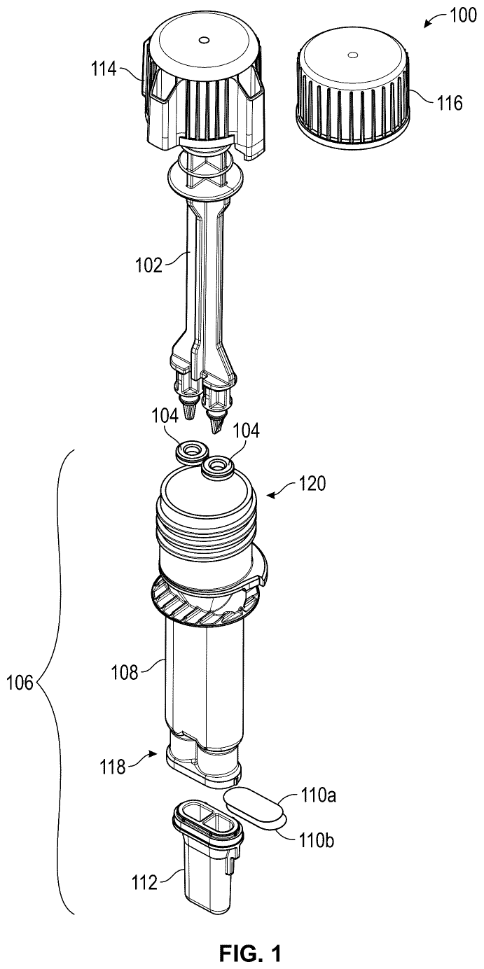

An example diagnostic test device 100 according to the present disclosure is now described with reference to FIGS. 1-7E.

The diagnostic test device 100 is implemented in a rapid, nucleic acid-based test system capable of performing automated molecular diagnostic testing for the detection of a variety of analytes of interest. The diagnostic test device 100 includes a cartridge 106 that is configured to be inserted into a diagnostic instrument of the test system. In one non-limiting example, the cartridge 106 is a consumable plastic container. The cartridge 106 can be formed of an injection-molded plastic, or any other suitable material. The cartridge 106 may include a barcode, for example a barcode displayed on an exterior surface of the cartridge 106, which can be scanned by the diagnostic test apparatus to automatically identify the assay to be performed on a patient sample that is added to the cartridge 106. In this non-limiting example, the assay includes a sample preparation assay and an isothermal amplification assay for the detection of nucleic acids of interest. A user may enter patient and/or sample information via a touchscreen on the instrument or via a barcode scan.

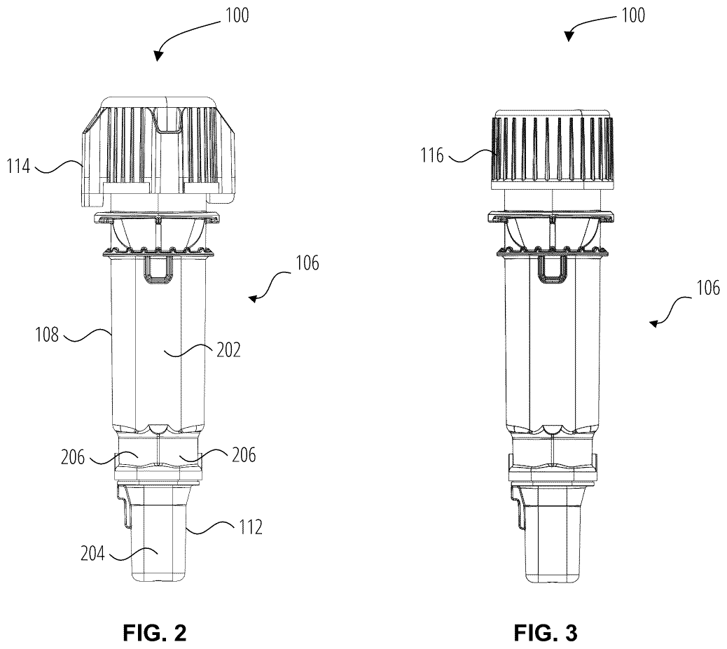

In addition to the cartridge 106, the diagnostic test device 100 includes a dispensing mechanism 102 that is configured to interface with the cartridge 106 as illustrated in FIG. 1. The cartridge 106 may include a cartridge body 108, a test container 112, and one or more seals 110a and 110b. The diagnostic test device 100 may include a closure configured to close a first end 120 of the cartridge body 108. For example, the diagnostic test device can include a dispense cap 114 and/or a transportation cap 116. The dispense cap 114 may be coupled to the dispensing mechanism 102. The dispensing mechanism 102 may include one or more sealing members 104, for example an o-ring, gasket, or a grommet. In the non-limiting embodiment of FIG. 1, the one or more sealing members 104 include two o-rings. As illustrated in FIGS. 2 and 3, the dispense cap 114 and the transportation cap 116 are each configured to be attached to the first end 120 of the cartridge body 108 to close or seal the first end 120. In one example, the transportation cap 116 is configured to reversibly close or seal the first end 120, and the dispense cap 114 is configured to irreversibly close or seal the first end 120. The cartridge body 108 includes a sample preparation reservoir 202 and one or more cylindrical chambers 206. The cartridge body 108 can form the sample preparation reservoir 108 and the one or more cylindrical chambers 206. Material, such as a fluid, present in the sample preparation reservoir 202 and the one or more cylindrical chambers 206 can be enclosed within the cartridge 108. The test container 112 includes one or more diagnostic test reservoirs 204. The test container 112 can form the one or more diagnostic test reservoirs 204. Material, such as a fluid, present in the one or more diagnostic test reservoirs 204 can be enclosed within the test container 112.

The test container 112 of the cartridge 106 can take any suitable shape and size. In the non-limiting embodiment of FIGS. 1-7, the test container 112 includes one or more tubes, where each tube forms a single diagnostic test reservoir 204. It will be understood, however, that other configurations can be suitably implemented.

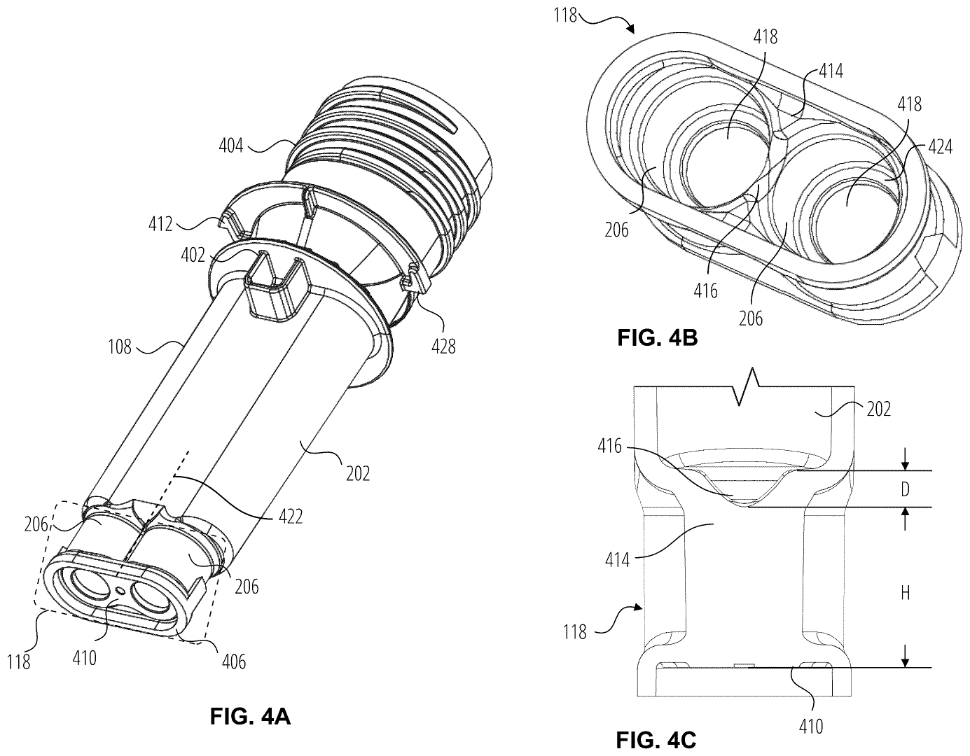

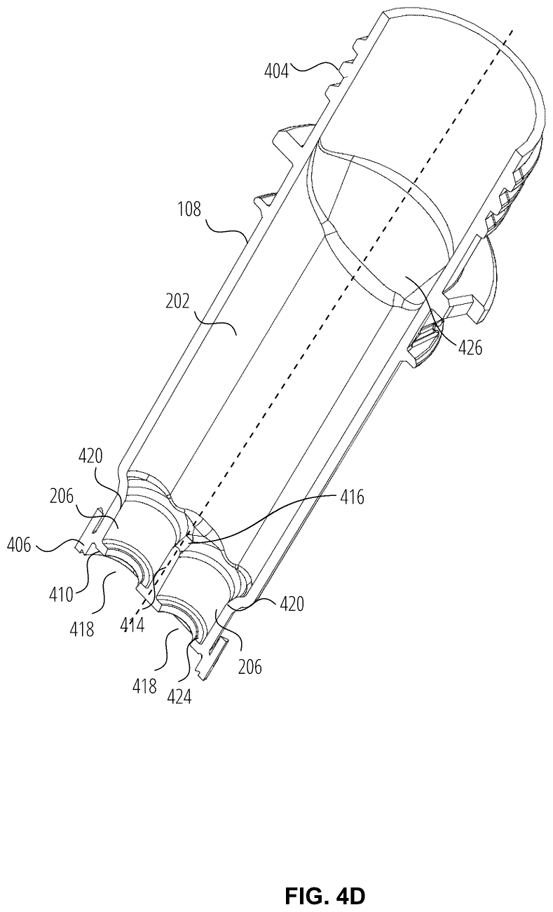

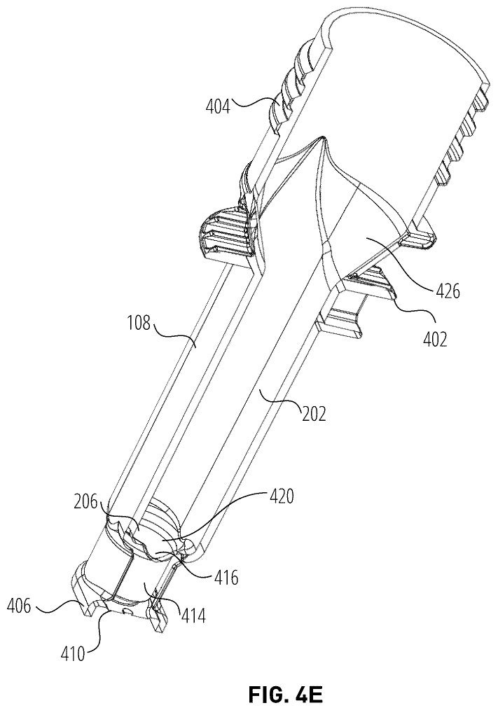

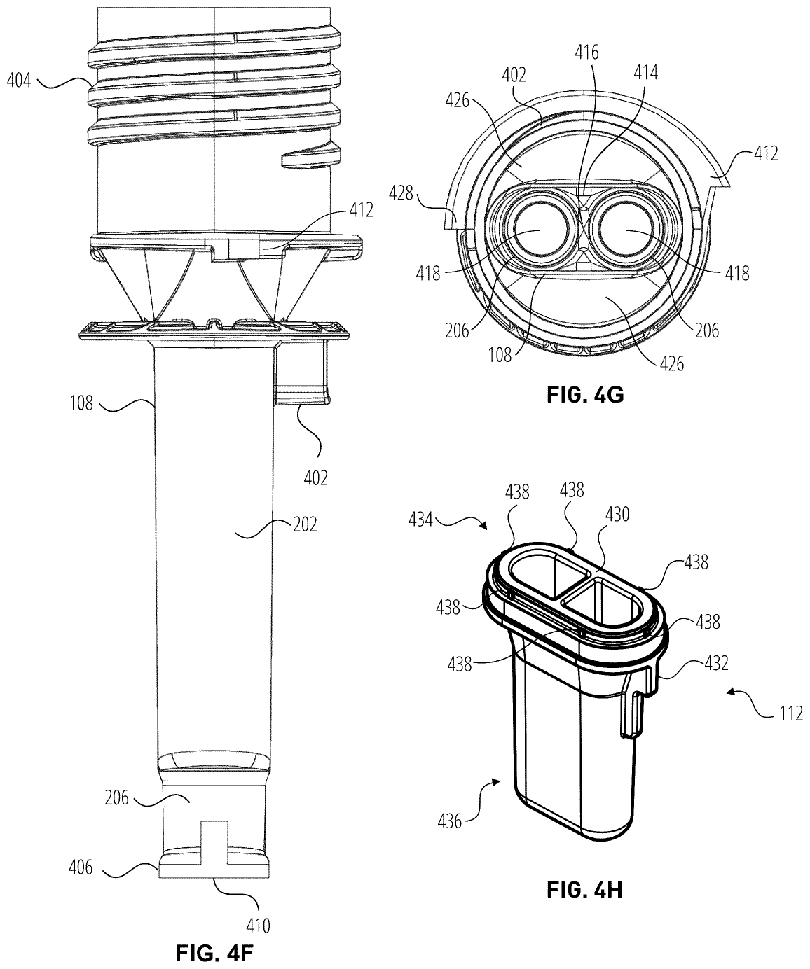

FIG. 4H illustrates a view of the test container 112. Amplification of an analyte of interest may occur within the one or more diagnostic test reservoirs 204 of the test container 112. When attached to the cartridge body 106 with seals 110a and/or 110b, the one or more diagnostic test reservoirs 204 are physically and fluidically separate from each other. A second end 118 opposite the first end 120 of the cartridge body 108 may be coupled to the test container 112. Other configurations can be suitably implemented. For example, in another non-limiting embodiment, the cartridge body 108 and the test container 112 are integrated in a unitary structure. Amplification, such as but not limited to isothermal amplification, and detection, such as but not limited to fluorescence detection, of one or more analytes of interest may take place in the one or more diagnostic test reservoirs 204. Optical signals can be directed to the one or more diagnostic test reservoirs 204, and optical signals emitted from the one or more diagnostic test reservoirs 204 can be detected and correlated to the presence, absence, and, in some cases, quantity of the one or more analytes of interest may be directed to the one or more diagnostic test reservoirs 204. The walls of the test container 112 may include a plastic, for example a polypropylene material or any other suitable material (such as, but not limited to, polyethylene). It may be desirable to choose a material that is transparent or substantially transparent to facilitate the transmission of optical signals through the walls of the test container 112.

It is to be understood that the present disclosure is not limited to test containers 112 having two diagnostic test reservoirs 204 as depicted in FIGS. 1 and 4H. For instance, a test container 112 can be implemented with one diagnostic test reservoir 204. Alternatively, a test container 112 can be implemented with three, four, or more diagnostic test reservoirs 204.

The test container 112 may include a detection tab 432. The detection tab 432 may facilitate detection of the presence or the absence of the test container 112 received within the diagnostic test apparatus. For example, the diagnostic test apparatus can include a sensor, such as a mechanical sensor, configured to interact with the detection tab 432 of the test container 112. Insertion of the test container 112 into the diagnostic test apparatus can cause the detection tab 432 to press the mechanical sensor of the diagnostic test apparatus, indicating that the test container 112 is properly seated within the diagnostic test apparatus. Other configurations can be suitably implemented. For example, the diagnostic test apparatus may include an optical sensor that emits an optical signal that is interrupted by the detection tab 432 when the test container 112 is properly seated. The detection tab 432 may include a “stepped” shape as illustrated in FIG. 4H. The test container 112 may also include a lip 430 to facilitate attachment of the test container 112 to the cartridge body 108 as described in greater detail below.

The one or more diagnostic test reservoirs 204 can be pre-loaded with reaction components to run a specific diagnostic test. For example, the one or more diagnostic test reservoirs 204 may contain lyophilized reagents. The lyophilized reagents may include enzymes, primers, probes, beacons, salts, and/or other reagents used in assay reactions. Mixing beads may also be included within the one or more diagnostic test reservoirs 204. The beads can be magnetic beads. The beads may be embedded inside a pellet of lyophilized reagents. When a fluid sample is introduced into the one or more diagnostic test reservoirs 204 and rehydrates the lyophilized reagents, the beads may facilitate mixing of the lyophilized reagents with the fluid sample. For example, the beads may be moved within the one or more diagnostic test reservoirs 204 under the influence of a magnetic force, to cause motion within any liquid within the one or more diagnostic test reservoirs 204 and aid in dissolving the lyophilized reagents. The bead may include stainless steel or any other suitable material. In alternative embodiments, the one or more diagnostic test reservoirs 204 can be pre-loaded with liquid reagents. In such embodiments, it may still be desirable to mix the preloaded liquid reagents with fluid sample, for example by agitating magnetic beads included within the one or more diagnostic test reservoirs 204.

FIGS. 4A-4G illustrate aspects of the cartridge body 108 according to the present disclosure. As shown in the cross-sectional view of FIG. 4D, the cartridge body 108 includes the sample preparation reservoir 202 and the one or more cylindrical chambers 206. The cartridge body 108 can also include a key 402, a threaded wall 404 at the first end 120, a lip 406 at the second end 118, a locking thread 412, and a blocking flange 428. An underside 410 of the one or more cylindrical chambers 206 are also illustrated in FIGS. 4A and 4D. The cartridge body 108 can also include a lower surface 424 of the cylindrical chamber 206. The sample preparation reservoir 202 can be pre-loaded with reaction components to run a specific diagnostic test. For example, the sample preparation reservoir 202 may be pre-loaded with a volume of sample preparation fluid. A sample may be carried on a swab, which can be inserted into the sample preparation reservoir 202. The sample preparation reservoir 202 may contain a sample preparation fluid to wash the sample from the swab into the sample preparation fluid, thereby creating a fluid sample within the sample preparation reservoir 202. The fluid sample in the sample preparation reservoir 202 may be configured to undergo processing to collect and/or concentrate nucleic acids, such as DNA and/or RNA. In some embodiments, the sample preparation fluid may include an elution lysis buffer (ELB). As illustrative examples, ELB may include red blood cell lysis buffer (RBCC), glycine running buffer solution (GRBS), and/or sodium dodecylsulfate solution (SDS). In alternative embodiments, the sample preparation fluid need not be pre-loaded into the sample preparation reservoir 202, but may be loaded shortly before the sample is introduced.

FIG. 4B shows a top-down cross-sectional view of the second end 118, indicated by the dotted rectangle in FIG. 4A, of the cartridge body 108. FIG. 4C shows a side cross-sectional view from dotted line 422 shown in FIG. 4A.

The sample preparation reservoir 202 may have a fluidic volume many times larger than the one or more diagnostic test reservoirs 204. As an illustrative example, the sample preparation reservoir 202 may have a volume of about 6 mL while the one or more diagnostic test reservoirs 204 may contain a combined total fluidic volume of about 400 μL. In some examples, the sample preparation reservoir 202 may hold a fluidic volume of between 0 and 5 mL, between 0.5 and 4.5 mL, between 1 and 4.0 mL, between 1.5 and 3.5 mL, between 2 and 3 mL, or any value or range within or bounded by any of these ranges or values, although values outside these values or ranges can be used in some cases. Additionally or alternatively, in some examples the sample preparation reservoir 202 may hold between 1 and 3 mL of fluidic volume. The amount of sample preparation fluid actually held by the sample preparation reservoir 202 may depend on the particular assay.

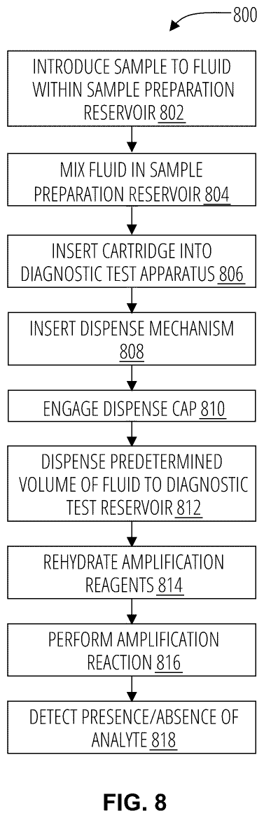

The one or more diagnostic test reservoirs 204 are configured to receive a predetermined volume of fluid sample from the sample preparation reservoir 202 through a process according to the present disclosure, including but not limited to the example process described below with reference to FIG. 8. The one or more diagnostic test reservoirs 204 may hold a fluidic volume of up to 50 μL of liquid, up to 75 μL of liquid, up to 100 μL of liquid, up to 150 μL of liquid, up to 200 μL of liquid, up to 250 μL of liquid, up to 300 μL of liquid, up to 350 μL of liquid, up to 400 μL of liquid, up to 450 μL of liquid, up to 500 μL of liquid, up to 1000 μL of liquid, or any value or range within or bounded by any of these ranges or values, although values outside these values or ranges can be used in some cases. Additionally or alternatively, in some examples the one or more diagnostic test reservoirs 204 may hold 200 μL of fluidic volume.

In some examples, the cartridge body 108 may include a geometry to facilitate rapid heating of the contents of the sample preparation reservoir 202. For example, the cartridge body 108 may have a relatively high surface area-to-volume ratio, which may facilitate rapid heating, for example by having an oblong cross-section. The walls of the cartridge body 108 may include a polypropylene material or any other suitable material (such as, but not limited to, polyethylene). In the embodiment depicted in FIGS. 4A-4G, the cartridge body 108 transitions from having a circular lateral cross-section near first end 120 to having an oblong lateral cross-section lower within the cartridge body 108. Depicted in FIGS. 4D, 4E, and 4G, a sloped wall 426 of the cartridge body 108 is the feature that transitions from a circular lateral cross-section to an oblong lateral cross-section. At the portion of the sloped wall 246 closest to first end 120, the lateral cross-section is relatively circular, becoming relatively oblong with proximity to the cylindrical chambers 206.

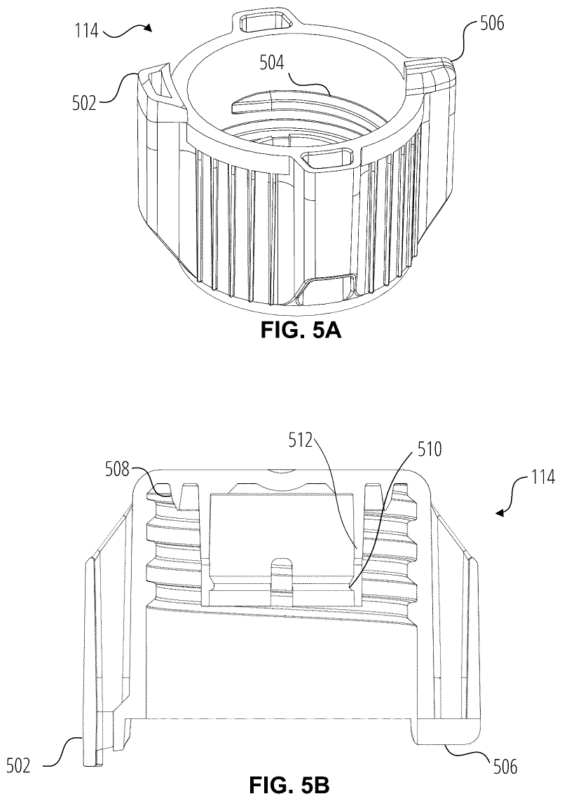

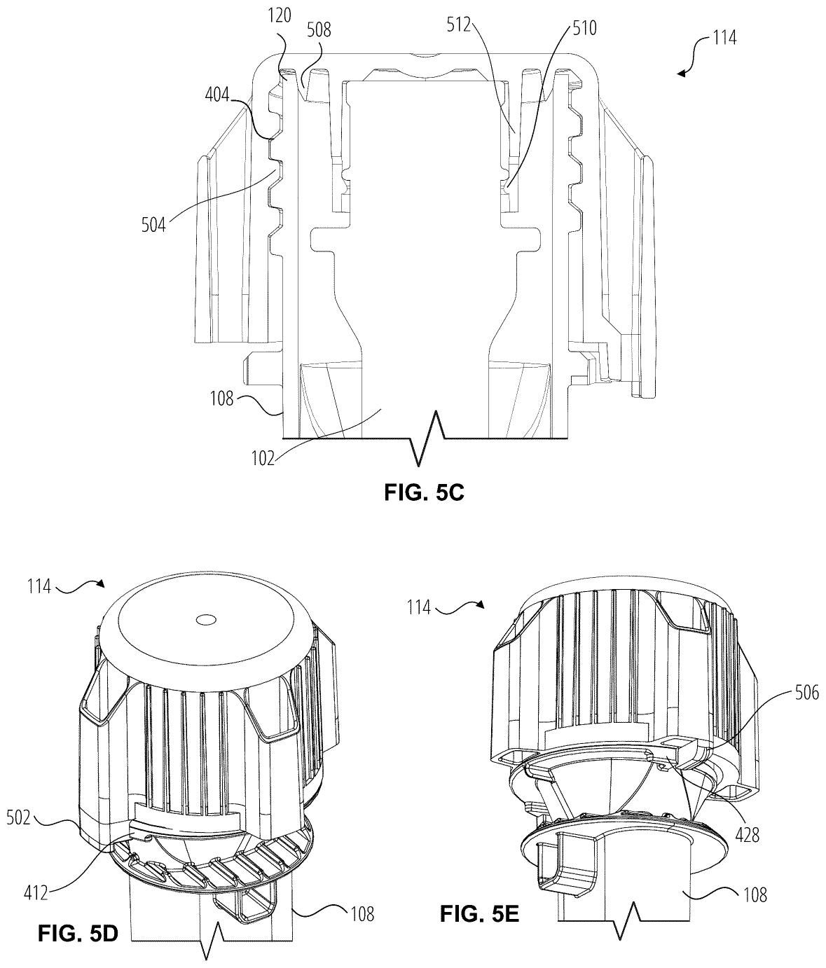

With reference now to FIGS. 5A and 5B, the dispense cap 114 includes a locking tab 502, internal threads 504, an overtravel tab 506, a plug seal 508, and an internal ring 510 projecting from a flange 512. The internal ring 510 in this non-limiting embodiment is a raised annular portion that projects from an internal surface of the flange 512. The flange 512 is configured to surround at least a portion of an end 606 of the dispensing mechanism 102 when the dispensing mechanism 102 is coupled to the dispense cap 114. The internal ring 510 engages a corresponding ring 610 at the end 606 of the dispensing mechanism 102 in a way that allows the dispense cap 114 to rotate freely about the dispense mechanism 102 when it is coupled to the end 606 of the dispense mechanism 102. In embodiments of the present disclosure, the dispense cap 114 rotates about a longitudinal axis of the dispensing mechanism 102 even though the dispense mechanism 102 remains stationary after insertion into the cartridge body 108, as described in greater below.

In this non-limiting example, the internal ring 510 and the ring 610 are reversibly coupled with a flexible interference fit, allowing the ring 610 to be reversibly snapped into and snapped out of the internal ring 510. It will be understood that other mechanisms to couple the dispense cap 114 to the dispense mechanism 102 can be suitably implemented.

The threaded wall 404 at the first end 120 of the cartridge body 108 may be engaged by a cap, for example the dispense cap 114 or the transportation cap 116. In embodiments of the cartridge body 108 which include the locking thread 412, the locking thread 412 may be also engaged by a cap, for example the dispense cap 114. Twisting a locking tab 502 of the dispense cap 114 in a first direction (in this example, a clockwise direction) past the locking thread 412 causes the dispense cap 114 to lock to the first end 120 of the cartridge body 108, thereby inhibiting and/or preventing motion of the dispense cap 114 in a second direction opposite the first direction (in this example, the counterclockwise direction). FIG. 5D shows the locking tab 502 in the locked position, positioned against the locking thread 412. In the locked position, the dispense cap 114 irreversibly engages the locking thread 412, such that a user cannot unthread the dispense cap 114 and uncouple the dispense cap 114 from the cartridge body 108. Advantageously, embodiments of the cartridge body 108 that include the locking thread 412 can completely seal the fluid sample inside the device 100 once the dispense cap 114 is engaged to the locking thread 412. This can prevent inadvertent contamination or leaking of the fluid sample 100 from the device 100 during or after a diagnostic test is performed.

As depicted in FIG. 5E, the blocking flange 428 of the cartridge body 108 may engage the overtravel tab 506 of the dispense cap 114. The blocking flange 428 may block the overtravel tab 506 from moving, and thereby prevent or inhibit the dispense cap 114 rotating further in a first direction (in this example, the clockwise direction). In some embodiments, the engagement of the overtravel tab 506 with the blocking flange 428 occurs when the dispense cap 114 is at substantially the same position as for the engagement of the locking thread 412 with the locking tab 502. That is to say, in some embodiments, the locking thread 412 and blocking flange 428 may each engage the locking tab 502 and the overtravel tab 506 respectively, such that the dispense cap 114 can neither twist counterclockwise nor twist clockwise once the dispense cap 114 has been twisted into the locked position. When engaged, the blocking flange 428 and the overtravel tab 506 prevent rotational motion of the dispense cap 114. When further rotation motion of the dispense cap 114 is prevented and/or inhibited, translational motion of the dispense cap 102 toward the end 410 of the cartridge body 108 is also prevented and/or inhibited. Advantageously, embodiments of the present disclosure allow translational motion of the dispense cap 102 toward the end 410 of the cartridge body 108 to be halted at a very precise, predetermined distance from the end 410 of the cartridge body, such that a precise, predetermined volume of fluid is consistently dispensed from the sample preparation reservoir 202 to the one or more diagnostic test reservoirs 204. In some non-limiting examples, further rotation of the dispense cap 114 another 7 to 10 degrees in the clockwise direction (past the locked position) resulted in an additional 10 μL of fluid being dispensed from the sample preparation reservoir 202 to the one or more diagnostic test reservoirs 204. In certain embodiments, an additional 10 μL dispense is a 10% error in volume dispersed. Accordingly, embodiments of the present disclosure that implement the blocking flange 428 and the overtravel tab 506 can consistently deliver a precise volume of fluid to the one or more diagnostic test reservoirs 204, thereby increasing reliability and accuracy of the test.

In some embodiments of the present disclosure, the cartridge body 108 includes features that are advantageously positioned to improve moldability and manufacturability of the cartridge body 108. In one non-limiting example, the protrusion of the cartridge body 108 that includes the locking thread 412 and the blocking flange 428 extends around less than half of the circumference of the upper part of the cartridge body 108 (for example, approximately 170° of the circumference). In some other embodiments, the protrusion may encompass substantially more or less of the circumference of the upper part of the cartridge body 108, for example, 330° of the circumference or 45° of the circumference. In embodiments where the cartridge body 108 is a single molded plastic piece, a protrusion that encompasses less than 180° of the circumference of the cartridge body 108 may possess better moldability. This is because, in such non-limiting examples, the protrusion on which the locking thread 412 and the blocking flange 428 are positioned does not cross a parting line used during manufacturing (for example during an injection molding process).

Referring to FIGS. 5B and 5C, the plug seal 508 of the dispense cap 114 is an annular flange extending from a top interior surface of the dispense cap 114. When in a locked position, the plug seal 508 may engage the first end 120 of the cartridge body 108. For example, an outer surface of the plug seal 508 can directly contact an inner surface of the first end 120 of the cartridge body 108, as illustrated in FIG. 5C. The plug seal 508 can prevent fluid within the cartridge body 108 from exiting or leaking out of the sample preparation reservoir 202 through the first end 120. Additionally, the plug seal 508 can prevent and/or inhibit fluid within the sample preparation reservoir from contacting the threaded wall 404 and/or threads 504. Accordingly, embodiments of the present disclosure implementing a dispense cap 114 with a plug seal 508 can advantageously reduce or eliminate risk that the external environment will be contaminated with fluid in the sample preparation reservoir 202, which could include pathogens.

In embodiments of the cartridge body 108 that include the key 402, the key 402 may engage the diagnostic test apparatus. The key 402 may help a user orient the cartridge 106 correctly within the diagnostic test apparatus. Additionally or alternatively, the key 402 may be sensed by the diagnostic test apparatus to indicate insertion of the cartridge 106.

An interior surface or wall 420 near the bottom of the cartridge body 108 may be shaped to define sides of at least one chamber, for example cylindrical chamber 206. In the embodiment illustrated in FIGS. 4A-4G, the sides of the cylindrical chambers 206 are formed by the interior surface 420. While the cartridge body 108 of illustrated embodiments includes two cylindrical chambers 206, other embodiments may include one or more cylindrical chambers 206. The cylindrical chambers 206 may include openings 418. Such openings facilitate dispense of liquid from the sample preparation reservoir 202 to the one or more diagnostic test reservoirs 204. The openings 418 may be covered by the seal 110a. In certain embodiments, each opening 418 may be covered by its own seal 110a, such that there is one seal 110a for each opening 418.

It is to be understood that the cartridge body 108 of the present disclosure is not limited two cylindrical chambers 206 as depicted in FIGS. 1-3, 4A-4G, and 7A-7E. For instance, a cartridge body 108 can be implemented with one cylindrical chamber 206. Alternatively, a cartridge body 108 can be implemented with three, four, or more cylindrical chambers 206. The number of cylindrical chambers 206 of the cartridge body 108 may correspond to the number of diagnostic test reservoirs 204 of the test container 112.

The seals 110a and 110b may include a foil material, and may be pierced by an application of mechanical force. The seals 110a and 110b need not be of the same material, but in some embodiments they may be of the same material. A seal 110b may be affixed to the test container 112 to cover the opening at first end 434 of the sample preparation reservoir 202, separating the sample preparation reservoir 202 and the one or more diagnostic test reservoirs 204. In one non-limiting embodiment, when the cartridge body 108 and test container 112 are joined into a single cartridge 106, the two seals 110a and 110b are pressed together. It may be desirable to attach the seal 110b to the top of the test container 112. In certain embodiments where the test container 112 holds lyophilized reagents, for example, the attachment of seal 110b can ensure moisture and/or other potential contaminants do not enter the test container 112 before the test container 112 and the cartridge body 108 are joined. Presence of moisture and/or contaminants within the test container 112 could lead to inaccurate assay results, for example false positives or false negatives. When affixed to underside 410 of the one or more cylindrical chambers 206, the seal 110a can hold fluid, for example liquid buffer, within the sample preparation reservoir 202 and cylindrical chambers 206. As illustrative examples, the seal 110a may be attached to the underside of the cylindrical chambers 206 by heat sealing, and the seal 110b may also be attached to the top of the test container 112 via heat sealing.

In some embodiments, there may be only one of either seal 110. In such embodiments, the seal 110 may be attached to cover the openings 418 of the cylindrical chambers 206 or the seal 110 may be attached to cover the first end 434 of the test container 112 before the test container 112 and the cartridge body 108 are joined. In such embodiments, the single seal 110 may keep fluid in the sample reparation reservoir 202 separate from the one or more diagnostic test reservoirs. Similarly, the single seal 110 may keep any lyophilized reagent within the diagnostic test reservoir 204 separate from the sample preparation reservoir 202.

In the example device 100, the cartridge body 108 is coupled to the test container 112 during manufacture and assembly of the device 100 prior to operation by an end user. Other embodiments can be suitably implemented. For example, in another non-limiting embodiment, the device 100 is formed of a single unitary structure that includes the cartridge body 108 integrally formed with the test container 112. In yet another non-limiting embodiment, the cartridge body 108 and the test container 112 are transported separately to an end user, and the end user couples the cartridge body 108 and the test container 112 prior to operation.

The cartridge body 108 can connect to the test container 112 using any number of coupling mechanisms, such as but not limited to a lip 406 that matingly connects to the lip 430 on the exterior surface of the test container 112. The sample preparation reservoir 202 and the one or more diagnostic test reservoirs 204, with the seal 110a and/or 110b therebetween, may be joined to form a cartridge 106. Thus, the seal 110a may define a bottom of the two cylindrical chambers formed by the interior surface 420. The seal 110b may define the top of the two diagnostic test reservoirs 204. Joining of the sample preparation reservoir 202 and the one or more diagnostic test reservoirs 204, with the seal 110a therebetween, to form a single joined structure may be accomplished by, for example, ultrasonic welding, glue, a snap-fit connection, a combination of these, or any other suitable joining mechanism. It may be desirable that the sample preparation reservoir 202 and one or more diagnostic test reservoirs 204 are joined sufficiently strongly to resist a buildup of pressure within the cylindrical chambers 206 and/or the one or more diagnostic test reservoirs 204. In embodiments where the sample preparation reservoir 202 and the one or more diagnostic test reservoirs 204 are joined via ultrasonic welding, the test container 112 may include one or more projections 438. The one or more projections 438 may be spaced around an exterior surface of the lip 430. The one or more projections 438 may aid in aligning the test container 112 against the end 410 and lip 406 of the second end 118 of the cartridge body 108 during ultrasonic welding. The one or more projections 438 may aid in centering the test container 112 relative to the lip 406 of the second end 118 of the cartridge body 108 during ultrasonic welding. For example, the one or more projections may ensure that the test container 112 is approximately or substantially equidistant from the edges of lip 406. The one or more projections 438 may thereby improve consistency and/or strength of the ultrasonic weld.

FIG. 5 illustrates the dispense cap 114, which may be coupled to the dispensing mechanism 102 according to embodiments of the present disclosure. The dispense cap 114 may include threads 504 configured to engage with the threaded wall 404 of the cartridge body 108. Additionally, in some embodiments, the dispense cap 114 includes a locking tab 502, which may engage the locking thread 412 of the cartridge body 108. Once the locking tab 502 of the dispense cap 114 has been rotated past the locking thread 412, further rotation in either direction may be prevented or inhibited, and the dispense cap 114 may be locked to the cartridge body 108.

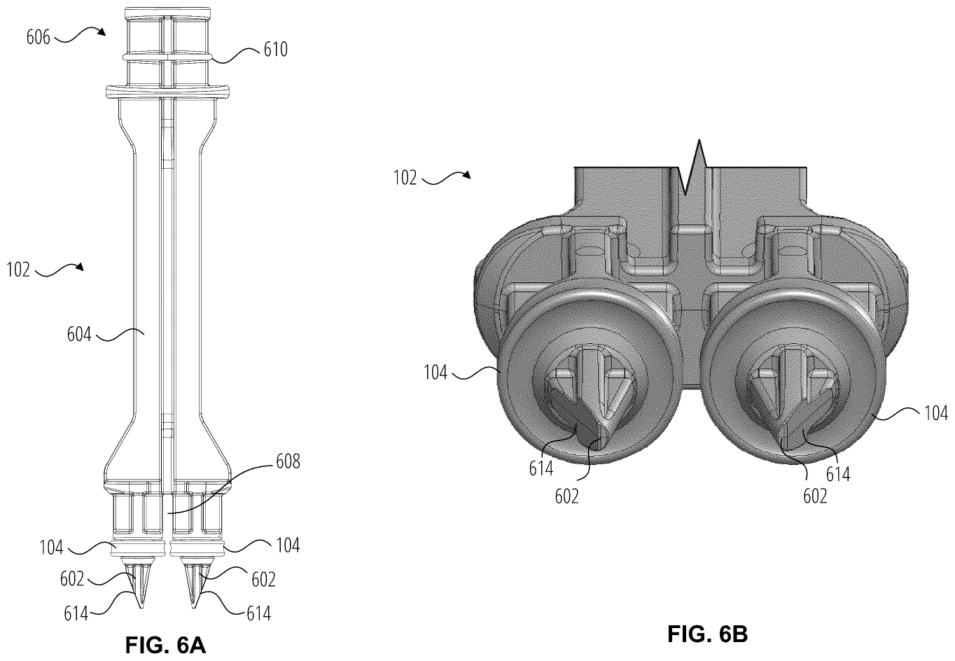

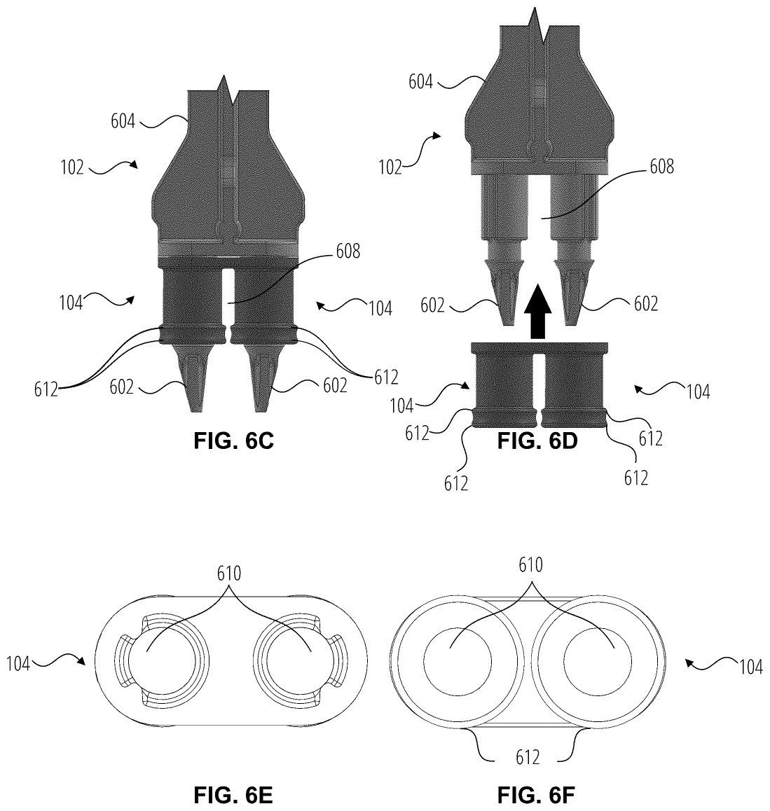

FIGS. 6A and 6B illustrate aspects of the dispensing mechanism 102. The dispensing mechanism 102 includes a shaft 604, one or more piercing members 602, and one or more sealing members 104. In embodiments of the present disclosure, the number of piercing members and sealing members 104 correspond to the number of cylindrical chambers 206 and the number of diagnostic test reservoirs 204. Accordingly, the piercing members 602, scaling members 104, cylindrical chambers 206, and diagnostic test reservoirs 204 are in a one-to-one correspondence.

The shaft 604 and the piercing member 602 of the dispensing mechanism 102 may include a plastic. The plastic may be, for example, a polycarbonate, an acrylonitirlie butadiene styrene (ABS), a nylon, another thermal plastic, a polypropylene material or any other suitable material (such as, but not limited to, polyethylene). The piercing member 602 may include a spike or other relatively sharp feature sufficient to pierce a seal, such as the seal 110a. In one example, the piercing member 602 includes a spiked rod. As illustrated in FIG. 6B, the exterior profile and the cross-section of the piercing member 602 may be in a cross-shape and/or a plus-sign shape. The piercing members 602 may include chamfered or beveled surfaces. For example, in the non-limiting embodiment of FIGS. 6A and 6B, a section of each of the piercing members 602 includes a chamfered surface 614. Cross and/or plus-sign shapes may facilitate flow of fluid past the piercing member 602 and through a pierced seal 110a, as fluid may flow more easily past the concave surfaces of the piercing member 602 while the chamfered surface 614 continues to enlarge the opening. Advantageously, the cross-shape or plus-sign shape of the piercing member 602 can form an opening in the seal 110a that has a shape and size that facilitates flow of fluid from the sample preparation reservoir 202 through the seal 110a. In one non-limiting example, the shape and size of the opening created in the seal 110a does not form leaves or sections of seal material that could obstruct or impede passage of the fluid through the opening.

In addition, the cross-shape or plus-sign shape of the piercing member 602 can advantageously allow air to leave the one or more diagnostic test reservoirs 204 and enter the sample preparation reservoir 202 before the opening is fully formed in the seal 110a. For example, air can travel past the concave surfaces of the piercing member 602 while the chamfered surface 614 continues to enlarge the opening. Pressure build-up in the one or more diagnostic test reservoirs 204 that would ordinarily act to impede flow of fluid into the one or more diagnostic test reservoirs 204 can thus be reduced as the opening is being formed. This is particularly advantageous in scenarios where air in the one or more diagnostic test reservoirs 204 is pressurized. It will be understood that the above-described advantages of embodiments of the piercing member 602 are also applicable to the formation of an opening in seal 110b.

It is to be understood that the dispensing mechanism 102 of the present disclosure is not limited two piercing members 602 as depicted in FIGS. 1, and 6A-7C. For instance, a dispensing mechanism 102 can be implemented with one piercing member 602. Alternatively, a dispensing mechanism 102 can be implemented with three, four, or more piercing members 602. The number of piercing members 602 of the dispensing mechanism 102 may correspond to the number of cylindrical chambers 206 of the cartridge body 108.

The dispensing mechanism 102 may include one or more sealing members 104, for example an o-ring, a gasket, or a grommet. The sealing member 104 may encircle at least a portion of the piercing member 602. FIGS. 6A and 6B illustrate embodiments where the sealing members 104 are o-rings. FIGS. 6C-6F illustrate an alternative embodiment wherein the sealing members 104 are one or more gaskets. FIGS. 6E and 6F are top and bottom views of an alternative embodiment of the sealing members 104. In certain embodiments where the sealing members 104 are gaskets, such as the one depicted in FIG. 6D, the sealing member may be formed separately from the dispensing mechanism 102 and coupled to the dispensing mechanism 102 by pushing the gasket sealing member 104 over the piercing members 602. In some embodiments, the gasket sealing member 104 may be overmolded onto the piercing members 602 during a process of manufacturing the piercing members 602. In the non-limiting example of FIGS. 6C-6F, the gasket is a unitary piece of material forming two channels 610, each channel 610 configured to receive one piercing member 602. The gasket also includes two annular portions 612 separated by a distance.

In one example where the piercing member 602 includes a spiked rod, the sealing member includes a sealing member 104 encircling the spiked rod. The sealing member 104 may be configured to directly contact the interior surface 420. In embodiments in which the sealing member 104 includes two o-rings, substantially the entire circumference of each o-ring can be in direct contact with the interior surface 420 of a cylindrical chamber 206 of the cartridge body 108. In some instances, such as the non-limiting example illustrated in FIGS. 6A and 6B, each o-ring includes two annular portions separated by a distance. Substantially the entire circumference of each annular portion 612 can be in direct contact with the interior surface 420 of a cylindrical chamber 206 of the cartridge body 108. In such instances, the presence of two independent annular portions can form a two-part seal against the interior surface 420, providing redundancy in the event one annular portion does not form an effective seal against the interior surface. In embodiments in which the sealing member 104 includes a gasket such as that illustrated in FIGS. 6C-6F, substantially the entire circumference of each annular portion 612 is in direct contact with the interior surface 420 of a cylindrical chamber 206 of the cartridge body 108. In such embodiments, the presence of two independent annular portions 612 separated by a distance can form a two-part seal against the interior surface 420, providing redundancy in the event one annular portion 612 does not form an effective seal against the interior surface 420.

The sealing member 104 may include an elastomeric material suitable for creating a liquid-impenetrable, or substantially liquid-impenetrable, seal when pressed against the material of the cartridge body 108. In some cases, the sealing member 104 includes a compressible material. In some non-limiting examples, the sealing member 104 includes a rubber, a butyl rubber, a thermoplastic vulcanizate (TPV), and/or a thermoplastic elastomer (TPE). In certain embodiments where the sealing member 104 is an o-ring, the sealing member 104 may include, for example, a 70 shore A butyl rubber. In certain embodiments where the sealing member 104 is a gasket (either a gasket that is formed separately before coupling to the dispense mechanism 102 or a gasket that is overmolded on the dispense mechanism), the sealing member 104 may include a 60 shore A TPV. It will be understood that many other materials can be suitably implemented in accordance with the present disclosure. The dispense cap 114 may be coupled to the dispensing mechanism 102. For example, the dispense cap 114 may be coupled to the dispensing mechanism 102 such that dispense cap 114 can rotate about the longitudinal axis of the dispensing mechanism 102. In one non-limiting embodiment, an end 606 of the dispensing mechanism 102 engages with an internal ring 510 in an interior top surface of the dispense cap 114 with a snap-fit mechanism that allows the dispensing mechanism 102 to rotate freely relative to the dispense cap 114.

In reference to FIGS. 4B and 4C, in embodiments having two or more cylindrical chambers 206, there may be a portion 414 of the interior surface that separates the two cylindrical chambers 206. The portion 414 of the interior surface between the two cylindrical chambers 206 may be formed to include a negative space, for example a notch 416. The notch 416 may, at least in part, define the predetermined volume of fluid that is dispensed from the sample preparation reservoir 202 to the one or more diagnostic test reservoirs 204. The depth of the notch 416 may thus be altered to tune the predetermined volume that is dispensed from the sample preparation reservoir 202 to the one or more diagnostic test reservoirs 204.

The predetermined volume of fluid that is dispensed to the one or more diagnostic test reservoirs 204 is defined by at least three variables: the radius of the cylindrical chamber 206, a height H of the cylindrical chamber 206 measured between the lower surface 424 and the lowest point of the notch 416, and the volume displaced by the piercing member 602. The depth of the notch 416, indicated by distance D in FIG. 4C, is inversely related to the predetermined volume that is dispensed because the depth of the notch 416 impacts the height H at which the one or more sealing members 104 engage the interior surface between the two cylindrical chambers 206. Three non-limiting examples are described below to illustrate the effect of the notch 416 on the predetermined volume of fluid that is dispensed. For purposes of these three examples, the only change in dimensions relating to the predetermined volume of dispensed fluid is to the depth D of the notch 416.

In a first non-limiting example illustrated in FIGS. 4B and 4C, the depth D of the notch 416 is approximately 0.2 mm for an embodiment where a volume of about 100 μL of fluid is dispensed from each cylindrical chamber 206 to a corresponding diagnostic test reservoir 204. In a second non-limiting example, the depth D of the notch 416 is approximately 0.1 mm, and a volume greater than about 100 μL of fluid is dispensed from each cylindrical chamber 206 to a corresponding diagnostic test reservoir 204. This is because, in this second non-limiting example, the depth D of the notch 416 is less than the depth D of the notch 416 in the first example. Stated another way, in the second example the one or more sealing members 104 engage the interior surface between the two cylindrical chambers 206 at a height H that is greater than the height H in the first example, thereby enclosing a larger volume of fluid within the two cylindrical chambers 206. In a third non-limiting example, the depth D of the notch 416 is approximately 0.4 mm, and a volume less than about 100 μL of fluid is dispensed to the one or more diagnostic test reservoirs 204. This is because, in this third non-limiting example, a depth D of the notch 416 is greater than a depth D of the notch 416 of the first example. Stated another way, in the third example the one or more sealing members 104 engage the interior surface between the two cylindrical chambers 206 at a height H that is less than the height H in the first example, thereby enclosing a smaller volume of fluid within the two cylindrical chambers 206. In some embodiments, the depth D of the notch 416 is between approximately 0 mm-2 mm, between approximately 0 mm-1.5 mm, between approximately 0 mm-1 mm, between approximately 0.1 mm-0.4 mm, though in some cases other values or ranges may be used. In a non-limiting example of the present disclosure in which approximately 100 μL of fluid is dispensed to each diagnostic test reservoir 204, the depth D of the notch 416 is between about 0.1 mm and about 0.4 mm.

In embodiments where the dispensing mechanism 102 includes two or more piercing members 602, the dispensing mechanism 102 may include a slot 608. The slot 608 is an empty space in the dispensing mechanism 102. The slot 608 may allow the one or more piercing members 602 and one or more sealing members 104 to pass beyond the portion 414 of the interior surface between the two cylindrical chambers 206.

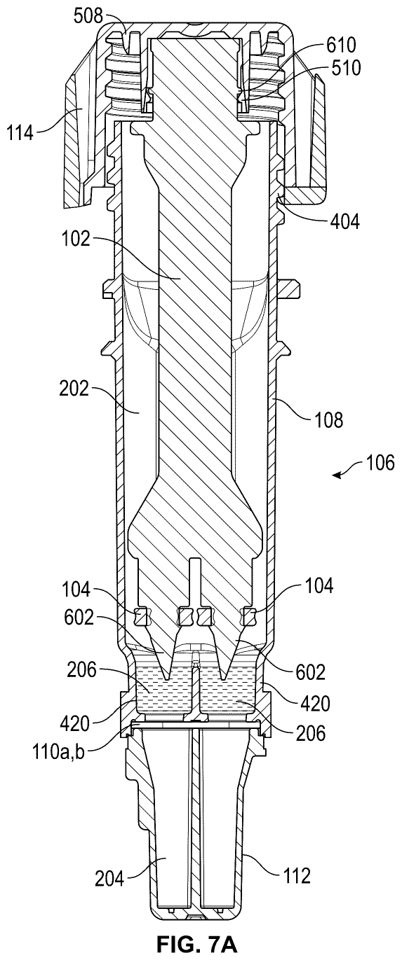

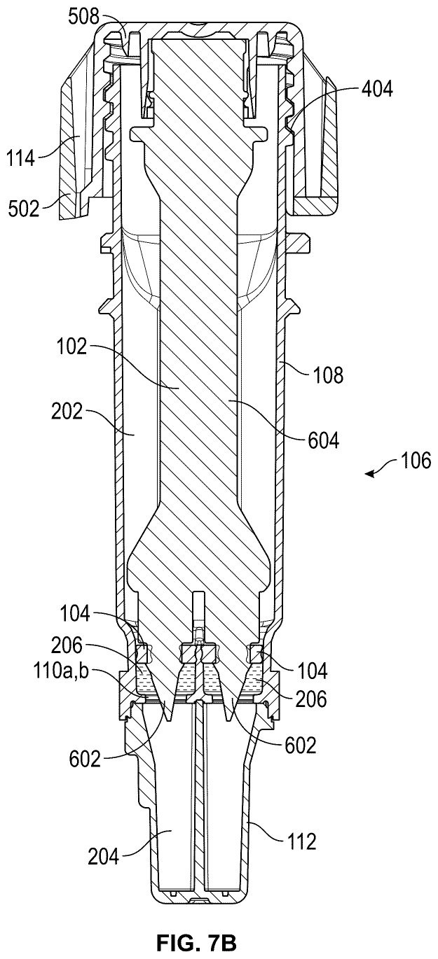

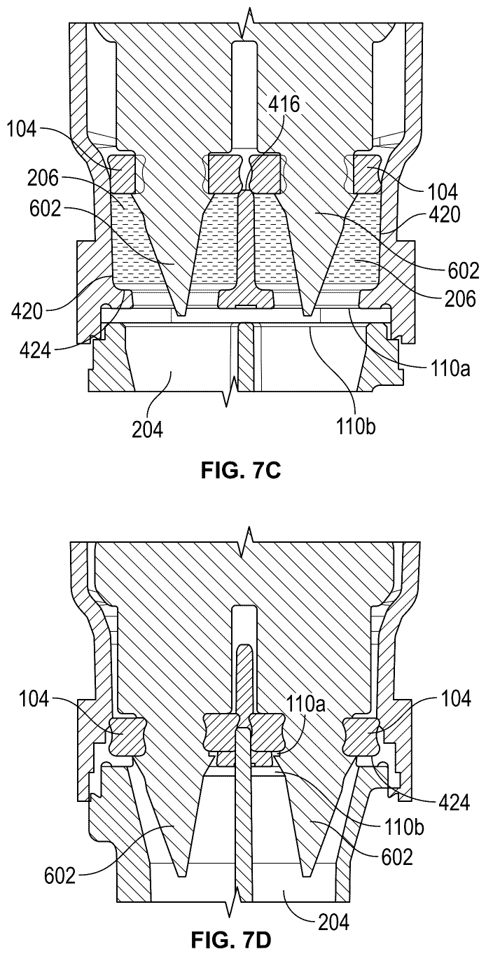

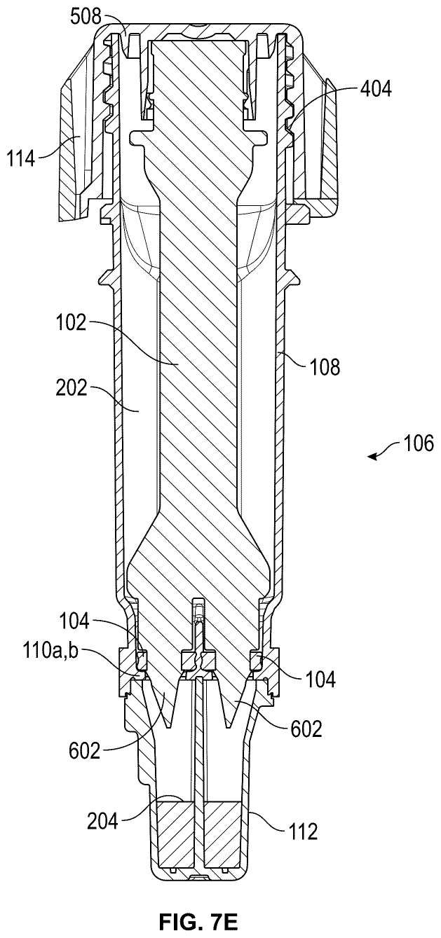

FIGS. 7A-7E illustrate four different positions of the dispensing mechanism 102 within the sample preparation reservoir 202 during a dispense operation according to the present disclosure. This action involves movement of the dispensing mechanism 102 relative to the cartridge body 108 to break the seal 110a and/or seal 110b, pushing a predetermined amount volume of fluid into the one or more diagnostic test reservoirs 204. Advantageously, embodiments of the devices, systems, and methods of the present disclosure dispense the predetermined amount of volume of fluid when one or more scaling members directly contact the interior surface defining the sides of the sample preparation reservoir 202 as the dispensing mechanism 102 translates along the longitudinal axis 108.

FIG. 7A illustrates the dispensing mechanism 102 inserted into the sample preparation reservoir 202, prior to engaging the threaded wall 404 with the dispense cap 114. The dispensing mechanism 102 can be placed in this position manually, by inserting the dispensing mechanism into the cartridge body 108 from above. The seals 110a and 110b are intact. As discussed, the dispensing mechanism 102 may be coupled to a dispense cap 114. The dispense cap 114 is configured to engage a threaded wall 404 of the sample preparation reservoir 202. The sealing members 104 are positioned above the cylindrical chambers 206 and have therefore not yet engaged the cylindrical chambers 206. However, as the sample preparation reservoir 202 is wider than it is deep (i.e. the top-down cross-section of the sample preparation reservoir is oblong), the interior of the sample preparation reservoir 202 orients dispensing mechanism 102 such that the piercing members 602 are substantially aligned with the cylindrical chambers 206 even when the piercing members 602 are positioned higher than the cylindrical chambers 206 within the cartridge body 108.

FIG. 7B illustrates the dispensing mechanism 102 after it has pierced the seal 110a and/or seal 110b. Relative to the position depicted in FIG. 7A, the dispensing mechanism 102 may be in the position of FIG. 7B after engaging the threaded wall 404 with the dispense cap 114 and continued twisting of the dispense cap 114. As discussed, the dispense cap 114 is coupled to the upper end 606 of the shaft 604 of the dispensing mechanism 102. This coupling allows the dispense cap 114 to rotate freely in relation to the shaft 604 and the piercing members 602. Thus, interaction of threaded wall 404 with threads 504 translates the twisting of the dispense cap 114 into vertical translational motion of the dispensing mechanism 102 along the longitudinal axis 108. As the cap 114 is tightened by clockwise twisting relative to the cartridge body 108, the dispensing mechanism 102 translates downward. In this position, the dispense cap 114 has not yet engaged the locking tab 502, and can still rotate with respect to the dispensing mechanism 102 and the cartridge body 108.

As illustrated in FIG. 7B, the dispensing mechanism 102 includes two sealing members 104 and two piercing members 602 configured to interact with the two cylindrical chambers 206 formed by the interior surface 420. When the dispense cap 114 is threaded on to the cartridge body 108 and twisted, the dispensing mechanism 102 is translated downward such that the piercing members approach and then break the seals 110a and 110b. This translation forces the piercing member 602 through the seal 110a.

There may be a buildup of pressure below the one or more sealing members 104, within the one or more diagnostic test reservoirs 204, as any air within the diagnostic test reservoirs 204 is compressed as the dispensing mechanism 102 translates downward. The threaded wall 404 and the threads 504 may be configured to resist upward force due to this buildup of pressure. Once the dispense cap 114 locks with the locking thread 412, the interaction between the locking thread 412 and the dispense cap 114 may resist upward motion as well. Additionally, the bond between the test container 112 and the cartridge body 108 should be sufficiently strong such that it does not break due to this buildup of pressure.

FIG. 7C depicts the dispensing mechanism 102 and the cartridge body 108 shortly after the piercing members 602 have pierced the seal 110a, but before fluid has travelled into the one or more diagnostic test reservoirs 204. The sealing members 104 have engaged the interior surface 420 of the cylindrical chambers 206. The lower face of each sealing member 104 has travelled below the notch 416, such that each sealing member 104 creates a complete seal with the interior surface 420 of the cylindrical chamber 206, preventing or substantially preventing any fluid from flowing relative to the sealing member 104; that is to say that fluid cannot flow from below the sealing member 104 to above the sealing member 104, and fluid cannot flow from above the sealing member 104 downward below the scaling member 104. Because the lowest point of notch 416 defines a height at which the top plane of the cylindrical chambers 206 can be defined, the predetermined volume is not defined until the lower face of the sealing members 104 have passed just below the notch 416. At the instant each piercing member 602 pierces the seal 110a, the predetermined volume (shaded area) is defined by the interior surface 420 of the cylindrical chamber 206, the sealing members 104, the piercing member 602, and the seal 110a. As the dispensing mechanism 102 translates further downward, the sealing members 104 and the piercing members 602 force the predetermined volume to flow through the pierced seal 110a into the diagnostic test reservoir 204.

As depicted in FIGS. 7D and 7E, the entire dispensing mechanism 102 translates downward relative to the cartridge body 108 until the predetermined volume, depicted as the shaded region at the bottom of the diagnostic test reservoirs 204 in FIG. 7E, has been fully and/or substantially dispensed into the diagnostic test reservoir 204. In some embodiments, there may be some volume of fluid remaining within the cylindrical chambers 206 when the dispense cap 114 locks. In some embodiments, the sealing members 104 may be flush against the lower surface 424 of the cylindrical chamber 206 once the predetermined volume has been dispensed into the diagnostic test reservoirs 204. In some embodiments, the sealing members 104 are in direct contact with the lower surface 424 of the cylindrical chamber 206 once the predetermined volume has been dispensed into the diagnostic test reservoirs 204. The dispense cap 114 is configured to lock against the locking thread 412 and the blocking flange 428 as the last of the predetermined volume is expelled from the one or more cylindrical chambers 206. This inhibits and/or prevents rotation of the dispense cap 114 relative to the cartridge body 108 and shaft 604, thereby preventing further and/or reverse translational motion of the dispensing mechanism 102 relative to the cartridge body 108. These and other features can allow embodiments of the diagnostic test reservoir 100 according to the present disclosure to accurately and consistently dispense no less than and no more than a predetermined volume of fluid into the diagnostic test reservoir,

Because the piercing members 602 and sealing members 104 are locked in place as described above, the predetermined volume dispensed to the one or more diagnostic test reservoirs 204 is locked within the test container 112. The piercing members 602 and sealing members 104 block passage of the predetermined volume of fluid from the diagnostic test reservoirs 204. Additionally, because the piercing members 602 and sealing members 104 are locked in place, no additional fluid nor other potential contaminants exterior to the diagnostic test device can enter the one or more diagnostic test reservoirs 204 or the sample preparation reservoir 202. As described below in reference to sample processing, the predetermined volume of fluid locked within the one or more diagnostic test reservoirs may undergo processing, for example thermal processing and/or optical processing. Such processing may assist in generating a result indicating the presence or absence of one or more target analytes within a sample introduced to the diagnostic test device 100.

### Sample Processing Using the Diagnostic Test Device

FIG. 8 illustrates an example process 800 of using a diagnostic device 100 in accordance with the present disclosure. The process can be implemented using illustrated embodiments, such as those depicted by FIGS. 1-7C and 9, as well as other embodiments in accordance with the present disclosure. In use, the cartridge 106 is provided with a transportation cap 116 engaging the threaded wall 404 of the cartridge body 108. The transportation cap 116 is removed from the cartridge body 108. For example, instructions for use of the device 100 can instruct the user to unthread the transportation cap 116 from the cartridge body 108. At block 802, the swab is inserted into the sample preparation reservoir 202 of the cartridge body 108 to deposit a sample. Instructions for use can instruct the user to swirl the tip of the swab in the sample preparation reservoir 202 according to a predefined protocol, for example a certain number of rotations and/or for a certain duration.

It will be understood that the sample can be dispensed into the sample preparation reservoir 202 using any suitable method. For example, a sample can be dispensed (such as by pipetting the sample) directly into the sample preparation reservoir 202 without the use of a swab. Liquid sample may include urine, blood, interstitial fluid, saliva, or any other suitable sample material. It will also be understood that embodiments of the present disclosure are not limited to liquid samples, and any suitable sample, including solid and gas samples, can be added to the sample preparation reservoir 202.

In this example implementation, the swab is then removed from the sample preparation reservoir 202 and disposed. The transportation cap 116 may then be threaded back onto the cartridge body 108. In another example, the transportation cap 116 is not threaded back onto the cartridge body 108.

The process next moves to block 804, where the cartridge 106, with the transportation cap 116 attached, is inverted or otherwise agitated to mix the fluid sample, dispersing the sample within the sample preparation fluid in the sample preparation reservoir 202. When the cartridge 106 is oriented such that the end 120 including the threaded wall 404 is pointed up (i.e., the cartridge 106 is not inverted), fluid sample may pool without air bubbles in the cylindrical chambers 206 of the sample preparation reservoir 202 under the influence of gravity. After mixing, It may be desirable that fluid sample pool in the cylindrical chambers 206 without air bubbles so that the intended volume of fluid can be dispensed to the sample preparation reservoirs 202. In embodiments where the transportation cap 116 is not re-engaged with the cartridge body 108, block 804 can include mixing the fluid sample without inverting the cartridge body 108.

In certain examples, the sample preparation fluid may be heated prior to introducing the swab and mixing the sample in the sample preparation fluid. In other examples, the sample preparation fluid is heated after mixing with the sample. In embodiments where the sample is added directly to the sample preparation reservoir 202, the sample may be added before or after heating the sample preparation solution. If present in the sample, particles containing analyte of interest may be lysed in the solution by the chemical action and/or elevated temperature of the sample preparation fluid.

The process next moves to block 806, where the cartridge 106 is inserted into the diagnostic test apparatus. The sample in the sample preparation fluid then undergoes processing. The transportation cap 116, if present, may be removed before or after the cartridge 106 is placed within the diagnostic test apparatus. The transportation cap 116 does not include a locking tab to engage the locking thread 412 of the cartridge body 108, and therefore cannot lock to the cartridge body 108 like the dispense cap 114.

The process next moves to block 808, where the dispensing mechanism 102 is inserted into the sample preparation reservoir 202. The dispensing mechanism 102 is lowered vertically through the sample preparation reservoir 202 toward the seal 110a, such that each piercing member 602 and sealing member 104 align or substantially align with a corresponding cylindrical chamber 206. Fluid can flow around and past the dispensing mechanism 102 as the dispensing mechanism 102 is lowered into the sample preparation reservoir 202.

The process then moves to block 810, where the dispense cap 114 of the dispensing mechanism 102 engages the sample preparation reservoir 202, in this example with the threaded wall 404 of the sample preparation reservoir 202.

Once the dispense cap 114 has descended down the threaded wall 404 a certain distance, the dispensing mechanism 102 will be at the position illustrated in FIG. 7C. The sealing members 104 engage the top of the two cylindrical chambers 206 formed by the interior surface 420 of the sample preparation reservoir 202. When the sealing members 104 engages the top of the two cylindrical chambers 206, the piercing member 602 is above the seal 110a, which separates the sample preparation reservoir 202 and the one or more diagnostic test reservoirs 204. The interior surface 420, the seal 110a, the sealing members 104, and the piercing member 602 together define a predetermined volume of fluid. In the illustrated embodiment, these features define two fluidically separated predetermined volumes of fluid, each volume associated with one of the two cylindrical chambers 206. The sealing members 104 form a fluid seal with interior surface 420, thereby trapping the predetermined volume(s) of fluid. Fluid present above the sealing members 104 within the sample preparation reservoir 202 cannot enter the two cylindrical chambers 206 after the sealing members 104 engage the interior surface 420.

In certain examples, the predetermined volume of fluid that is sealed in each cylindrical chamber 206 is up to 10 μL of liquid, 25 μL of liquid, up to 50 μL of liquid, up to 70 μL of liquid, up to 75 μL of liquid, up to 100 μL of liquid, up to 125 μL of liquid, up to 130 μL of liquid, up to 150 μL of liquid, up to 200 μL of liquid, up to 250 μL of liquid, up to 300 μL of liquid, up to 350 μL of liquid, up to 400 μL of liquid, up to 450 μL of liquid, up to 500 μL of liquid, up to 1000 μL of liquid, or any value or range within or bounded by any of these ranges or values, although values outside these values or ranges can be used in some cases. Additionally or alternatively, in some examples the predetermined volume of fluid is about 100 μL of liquid.

The dispense cap 114 is then further rotated to translate the dispensing mechanism 102 to reach the position illustrated in FIG. 7B. The piercing member 602 has pierced the seal 110a and the seal 110b between the sample preparation reservoir 202 and the one or more diagnostic test reservoirs 204. Further translation causes the sealing members 104 to slide along the interior surface 420 of the sample preparation reservoir 202, acting as a piston that forms a sliding seal with the two cylindrical chambers 206 of the sample preparation reservoir 202. For example, a liquid-tight seal can be formed between the sealing members 104 and the interior surface 420 as a result of a close fit between the sealing members 104 and the interior surface 420 inside the two cylindrical chambers 206 of the sample preparation reservoir 202.

The method next moves to block 812, where the seals 110a, 110b are pierced, and the predetermined volumes of fluid are pushed from within the cylindrical chambers 206 into the diagnostic test reservoirs 204 by the downward motion of the dispensing mechanism 102. This dispense action forces the predetermined volume of fluid into the diagnostic test reservoirs 204. The seals 110a, 110b ensure that there is no communication of fluid between the sample preparation reservoir 202 and the one or more diagnostic test reservoirs 204 until the dispensing action. Once the sealing members 104 have formed a seal with the interior surface 420, fluid in the sample preparation reservoir that is above the scaling members 104 is not dispensed into the one or more diagnostic test reservoirs 204. Thus, in embodiments of systems and methods of the present disclosure, a first portion of the total fluid volume that is present in the sample preparation reservoir 202 is dispensed to the diagnostic test reservoirs 204, while a second portion of the total fluid volume that is present in the sample preparation reservoir 202 is not dispensed to the diagnostic test reservoirs 204. In certain embodiments where there are two cylindrical chambers 206 each capable of dispensing about 100 μL, there may be a volume of 500 μL or more of fluid in the sample preparation reservoir 202. In certain embodiments where there are two cylindrical chambers 206 each capable of dispensing about 100 μL, there may be between 1 and 3 mL of fluid in the sample preparation reservoir 202. In some embodiments, the total fluid volume that is present in the sample preparation reservoir 202 is 1-300 times greater than the volume of the predetermined volume dispensed to the diagnostic test reservoirs 204. In some embodiments, the total fluid volume that is present in the sample preparation reservoir 202 is 5-50 times greater than the volume of the predetermined volume dispensed to the diagnostic test reservoirs 204. Ensuring that a consistent volume of fluid is dispensed into the diagnostic test reservoirs 204 may reduce variability of assay results. Consistent and reliable dispersion of fluid volume to the bottom of the diagnostic test reservoirs may also ensure a higher likelihood that sufficient sample material, for example genomic material, is available to the assay reaction to ensure an accurate test result.

In this non-limiting example of the present disclosure, the one or more diagnostic test reservoirs 204 includes two receiving chambers, together forming a test container 112. Each receiving chamber of the test container 112 is configured to align with a cylindrical chamber 206 of the sample preparation reservoir 202. The diagnostic test reservoir 204 can be heated to perform an amplification reaction in fluid dispensed into the diagnostic test reservoir 204. Optical fluorescence signals from the diagnostic test reservoir 204 can be detected through the walls of the test container 112.

In non-limiting embodiments of the present disclosure, the dispensing mechanism 102 freely moves along a longitudinal axis of the sample preparation reservoir 202, up until a point where a locking tab engages a locking thread, as described in further detail below. The fluid in the sample preparation reservoir 202 flows relative to the dispensing mechanism 102 as the dispensing mechanism 102 is lowered along the longitudinal axis of the sample preparation reservoir 202. In non-limiting examples of the present disclosure, the dispensing mechanism 102 and dispense cap 114 are the only movable components of the diagnostic test device 100 during operation by a user. The entire dispensing mechanism 102 translates in a single motion, downward along the longitudinal axis of the diagnostic test reservoir 204, until translation of the entire dispensing mechanism 102 is arrested as described above. The downward motion of the dispensing mechanism 102 first defines a predetermined volume of fluid bounded by the interior surface 420 of the sample preparation reservoir 202, the piercing member 602 of the dispensing mechanism 102, the sealing members 104 of the dispensing mechanism 102, and the seal 110a. The additional downward motion of the dispensing mechanism 102 next pierces the seals 110a, 110b with the piercing member 602. Further downward motion of the dispensing mechanism 102 finally dispenses the predetermined volume of fluid sample into the diagnostic test reservoir 204 through the piston action of the dispensing mechanism together with the seal formed with sealing members 104. One predetermined volume of fluid sample is thereby dispensed into a single diagnostic test reservoir 204.

Simultaneous with the downward motion of the dispensing mechanism 102 along the longitudinal axis of the sample preparation reservoir 202, rotation of the dispense cap 114 about the longitudinal axis of the sample preparation reservoir 202 causes the locking thread 412 of the cartridge 106 to engage with the locking tab 502 of the dispense cap 114. During the last rotation of the cap 114 to cause the piercing members 602 to pierce the seals 110a, 110b, the locking tab 502 on the dispense cap 114 rotates past the end of the locking thread 412. The locking thread 412 then substantially prevents and/or inhibits rotational motion of the dispense cap 114 in either direction, which in turn substantially prevents and/or inhibits translational motion of the dispensing mechanism 102. It may be desirable that the locking thread 412 locks to the top of the cartridge 106 so that the fluid in the sample preparation reservoir 202 remains sealed during and after a test operation. In addition, embodiments of this locking mechanism according to the present disclosure can advantageously lock the dispensing mechanism 102 in place to prevent any further movement of liquid and/or reagents between the sample preparation reservoir 202 and the diagnostic test reservoir 204.

In devices, systems, and methods according to the present disclosure, the dispensing mechanism 102 is a monolithic, single-piece structure that is the only movable component within the sample preparation reservoir 202, thereby reducing the possibility of alignment errors during sealing of the sealing members 104 and dispense of fluid into the diagnostic test reservoir 204. In embodiments of the present disclosure, the sealing members 104 easily align and reliably seat within the two cylindrical chambers 206. There is a single stroke motion that causes the downward translation of the dispensing mechanism 102, resulting in the dispense action. Advantageously, consistent and reliable sealing of the sealing members 104 during the dispense action contribute to a consistent and precise sub-volume of fluid in the sample preparation reservoir 202 being dispensed into the diagnostic test reservoir 204. This can advantageously contribute to more consistent and more accurate testing for the presence, absence, or quantity of an analyte of interest in the fluid that is dispensed into the diagnostic test reservoir 204.

The method next moves to block 814, where the predetermined volume of fluid dispensed into the diagnostic test reservoir 204 may rehydrate lyophilized reagents if present within the diagnostic test reservoir 204. The combination of the predetermined volume of the fluid and the rehydrated reagents within the diagnostic test reservoir 204 is referred to herein as the amplification fluid. It will be understood that embodiments of the present disclosure are not limited to rehydrating reagents using the dispensed fluid, or even to providing reagents in the diagnostic test reservoir 204. Accordingly, in some non-limiting embodiments, the composition of fluid dispensed into the diagnostic test reservoir 204 is the same as the composition of fluid that is tested for the presence, absence, or quantity of an analyte of interest in the diagnostic test reservoir 204.

Once reagents have been rehydrated, the method moves to block 816 where a reaction is performed in the amplification fluid in the diagnostic test reservoir 204. The reaction can include an amplification reaction. The reaction can include an assay. The reaction may involve applying heat to the diagnostic test reservoir 204, which is transferred to the fluid to facilitate an isothermal amplification reaction. In other cases, the amplification reaction includes cyclical heating to perform an amplification reaction. It will be understood that these example reactions and assays are not limiting and any suitable reaction can be performed in fluid in the diagnostic test reservoir 204.

The method ends at block 818, where the presence or absence of an analyte of interest is detected. The analyte of interest can be detected as the amplification reaction proceeds (for example, during a real-time PCR test) or at the termination of the amplification reaction. The presence or absence of an analyte may be detectable via a fluorescence signal generated during the amplification reaction, for example.

Methods of Using a Diagnostic Test Device with a Diagnostic Test Apparatus

In some examples, before or after the dispense action has occurred as described herein, the diagnostic test device 100 may be introduced into a diagnostic test apparatus 900. The device may be inserted into one or more heat blocks 902, 904 of the diagnostic test apparatus 900 configured to accept the diagnostic test device 100. A diagnostic test device 100 having a transportation cap 116 or a dispense cap 114 may be inserted into the diagnostic test apparatus 900.

In one non-limiting embodiment, the diagnostic test apparatus 900 applies heat using heat block 902 to the amplification fluid in the diagnostic test reservoir 204 to perform an amplification reaction. The diagnostic test apparatus 900 also directs optical signals to the diagnostic test reservoir 204, and receives optical signals from the diagnostic test reservoir 204 to detect an analyte of interest, if present, in the amplification fluid within the diagnostic test reservoir 204. The diagnostic test apparatus 900 may use one or more image sensors (not illustrated) to optically scan a portion of the test container 112, for example a bottom portion 436 of the test container 112. Such scanning may be used to detect and/or measure a positive control reporter within the amplification fluid. Measurement of the positive control reporter can confirm the dispensing action and that the amplification reaction is capable of proceeding as intended. Such scanning may also be used to detect and/or measure the progress of the test assay reaction. For example, the diagnostic testing apparatus 900 may optically scan the bottom portion of the diagnostic test reservoir 204 to detect and/or measure changes in fluorescence indicative of an ongoing amplification reaction due to the presence of an analyte. As described above, embodiments of the present disclosure are not limited to real-time detection during a reaction, and in some cases, detection is performed when the reaction is complete.

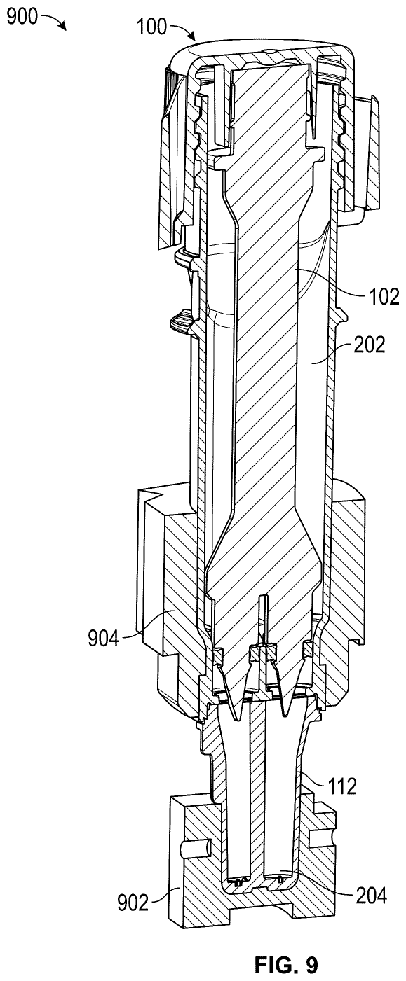

FIG. 9 illustrates a cross-sectional view of the diagnostic test device 100 received in one or more heat blocks 902, 904 of the diagnostic testing apparatus 900. The diagnostic test device 100 includes the dispensing mechanism 102 received in the sample preparation reservoir 202. In this non-limiting example, the test container 112 is received in a first heat block 902 of the diagnostic testing apparatus 900, and the sample preparation reservoir 202 is received in a second heat block 904 of the diagnostic testing apparatus 900nt. The second heat block 904 can apply heat to the cartridge body 108 to facilitate preparation of a sample for an assay or reaction in a fluid in the sample preparation reservoir of the cartridge body 108. The fluid contained within the sample preparation reservoir 202 may be heated as the cartridge body 108 is heated. After a sub-volume of the fluid in the sample preparation reservoir 202 is dispensed into the test container 112, the heat block 902 can apply heat to the test container 112 to perform an amplification reaction in the amplification fluid present in the test container 112. Windows within the heat bock 902 (not illustrated in this cross-sectional view) can allow optical signals to be directed to the one or more diagnostic test reservoirs 204, and for optical signals to be received from the one or more diagnostic test reservoirs 204, to detect an analyte of interest, if present, in the amplification fluid.

One or more optical sensors incorporated within the diagnostic testing apparatus 900 can capture fluorescence signals emitted from the amplification fluid during or after the amplification reaction. The digital output from the one or more image sensors can be used to confirm the test assay progression and confirm the correct release and flow of test reagents within the cartridge such that the integrity of the test can be confirmed by the controller and used to improve the reliability and accuracy of the test result.

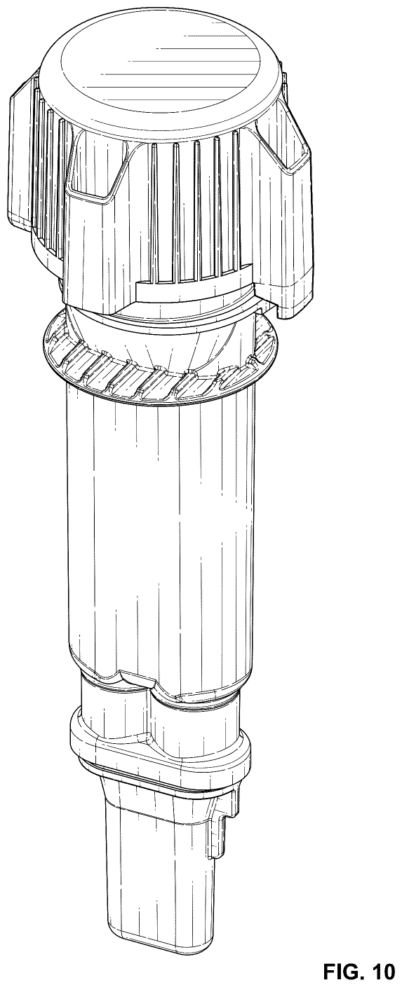

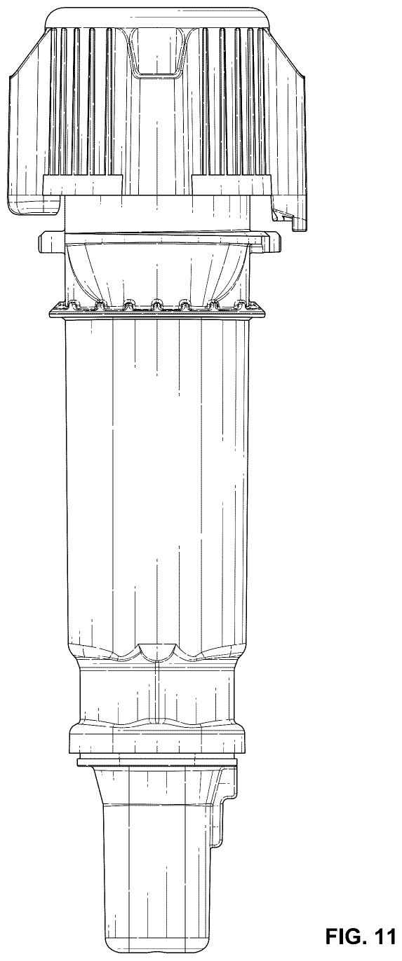

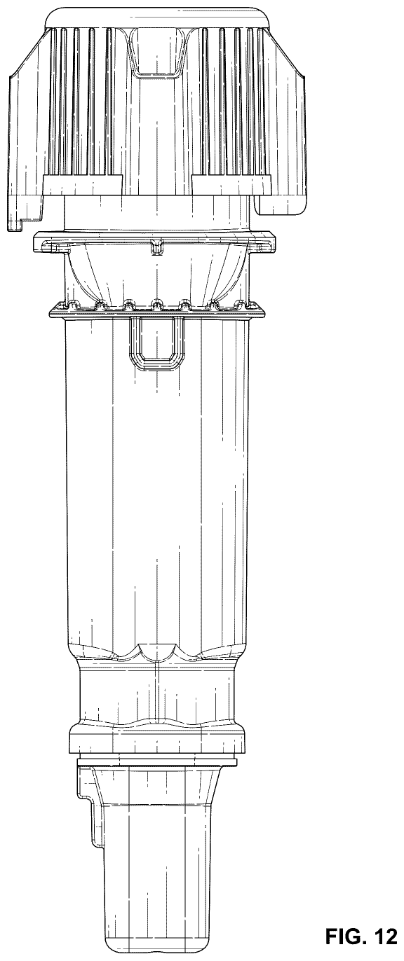

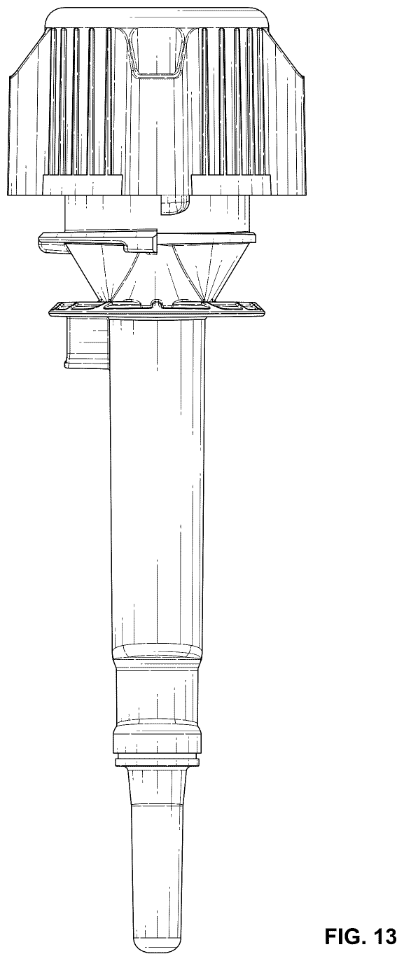

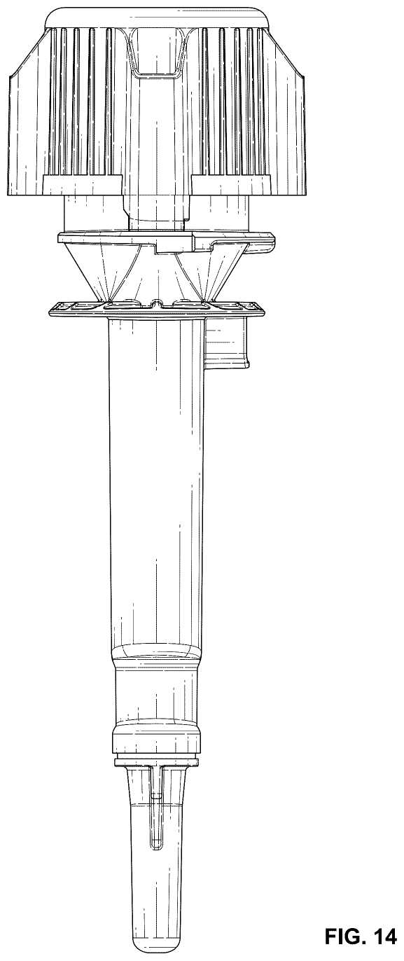

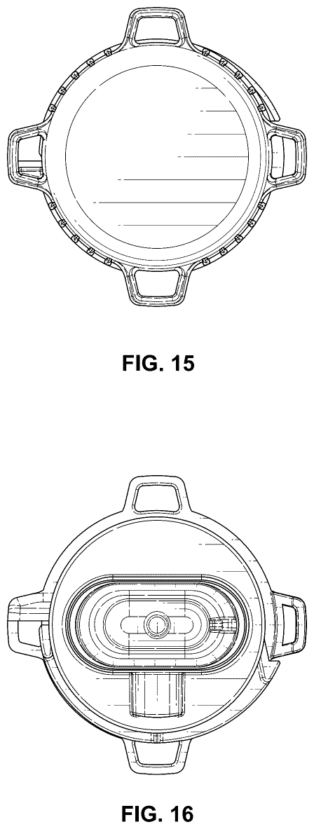

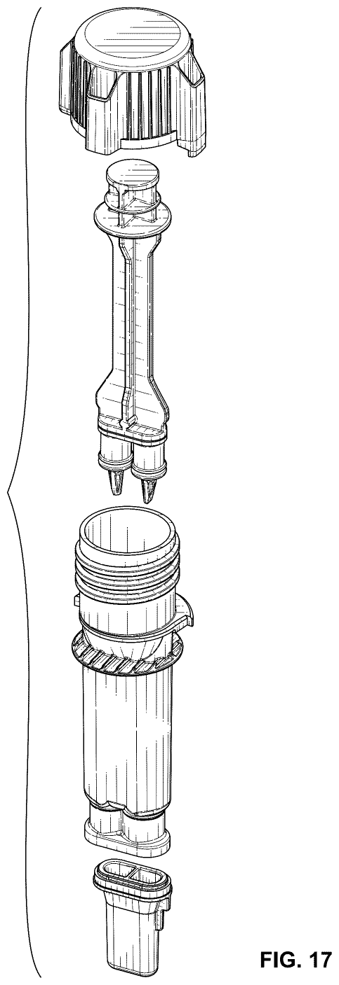

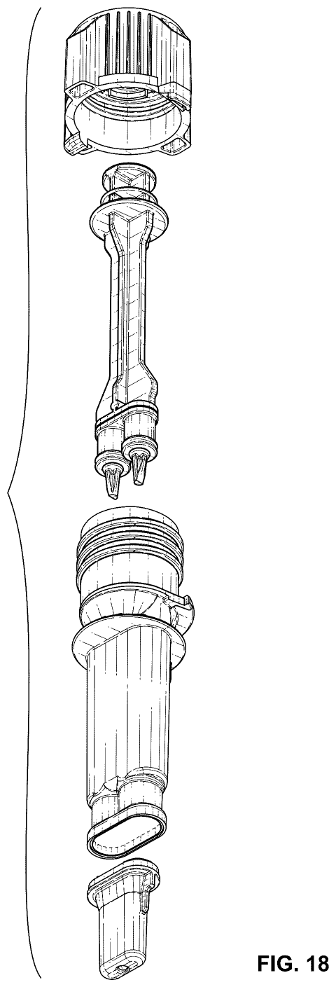

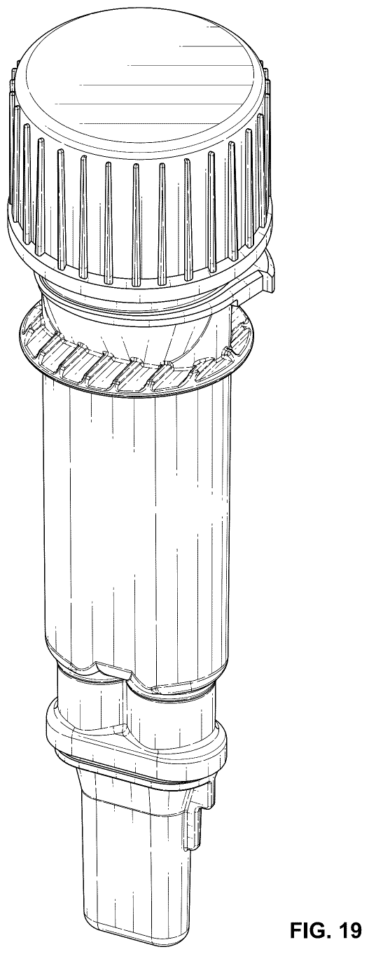

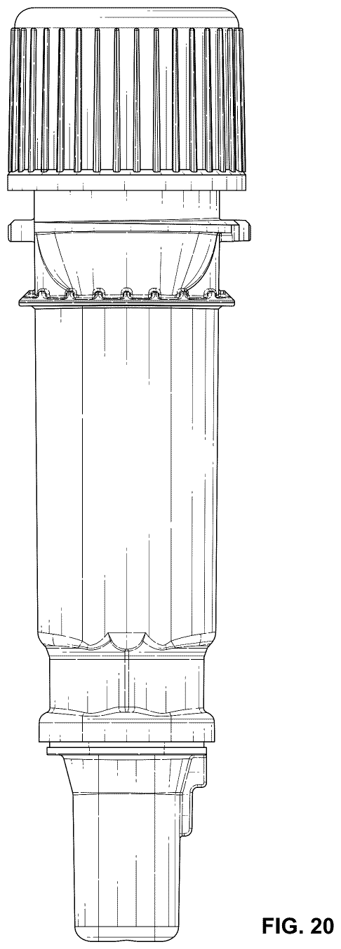

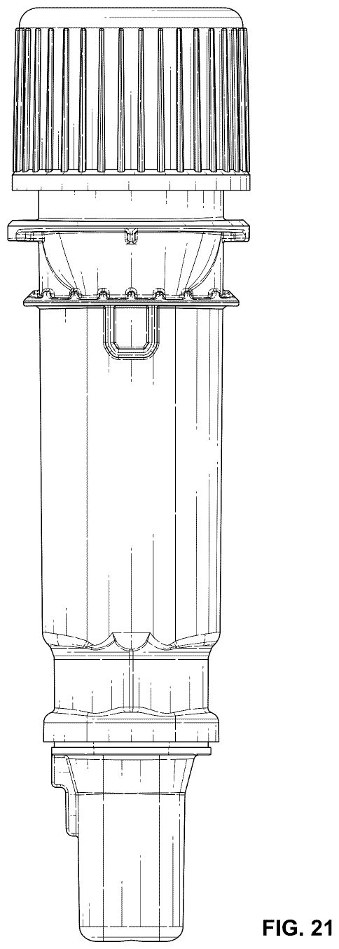

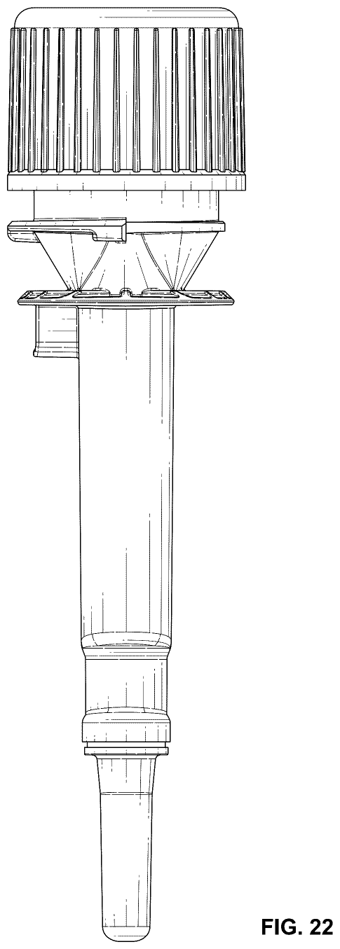

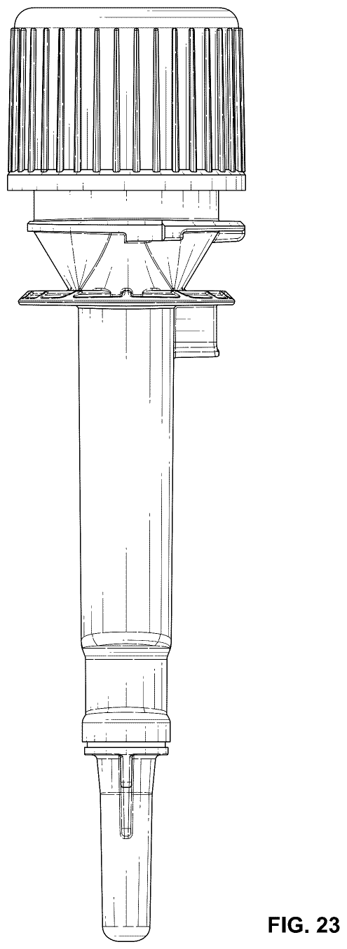

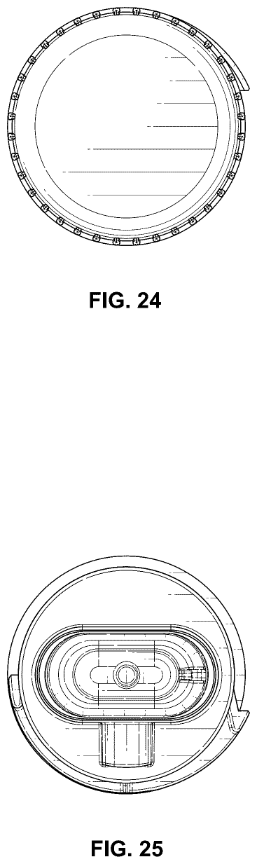

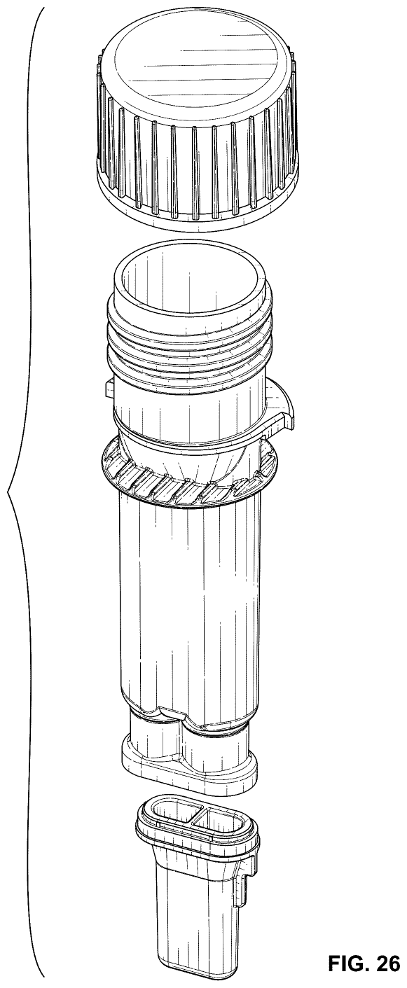

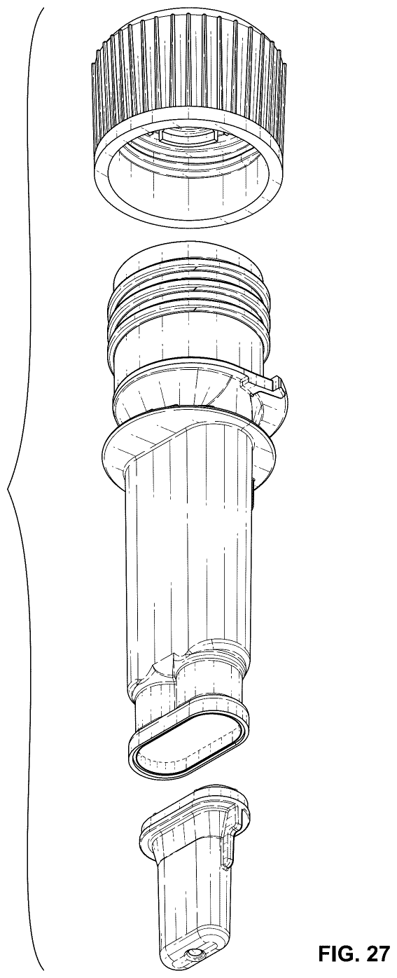

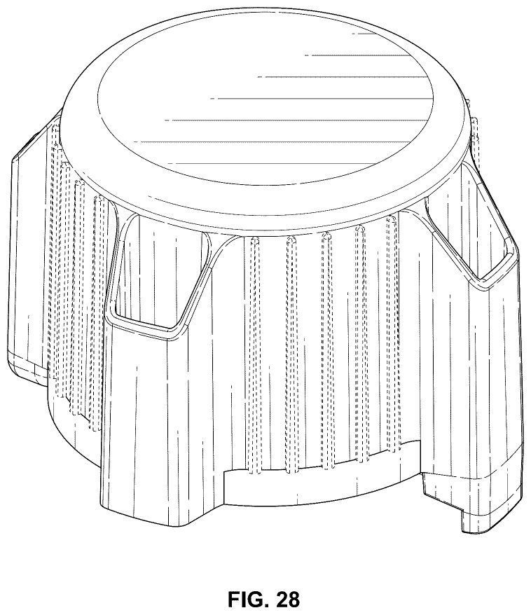

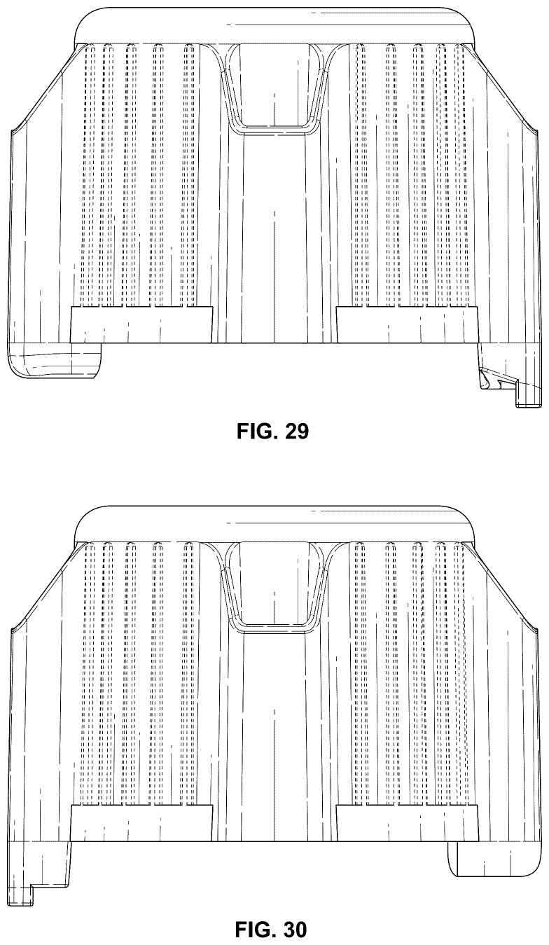

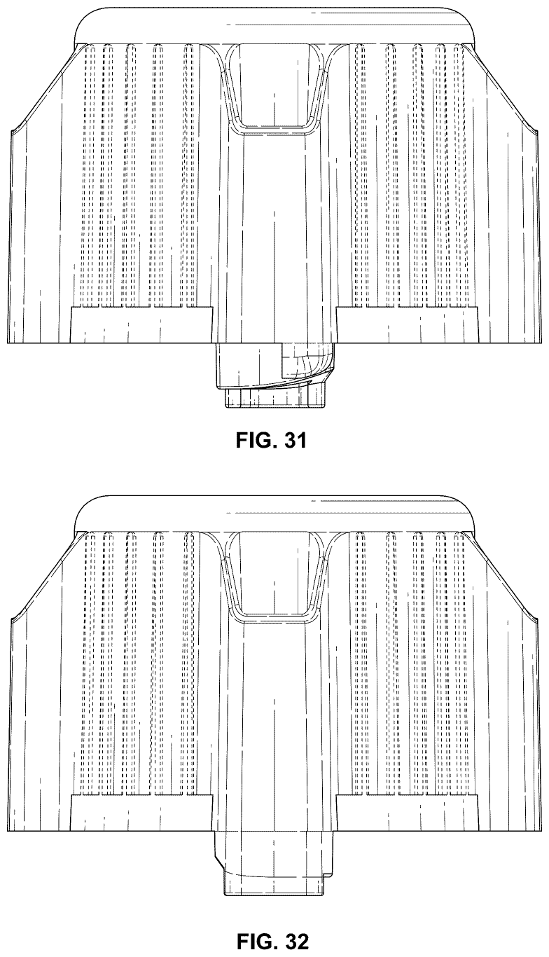

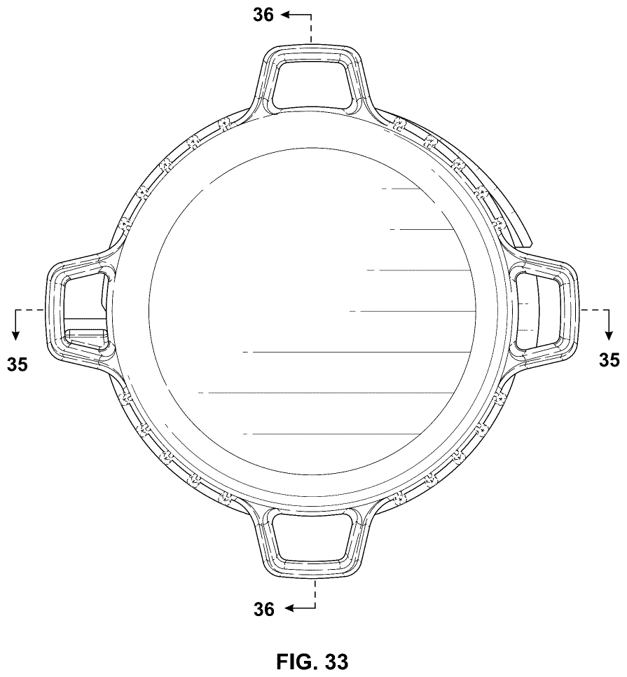

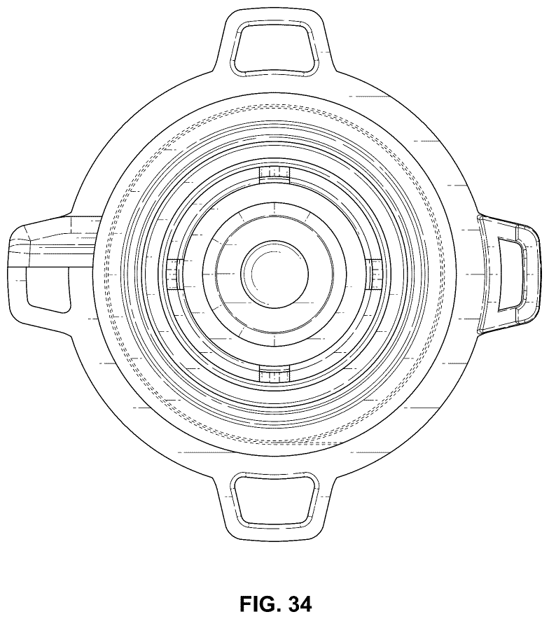

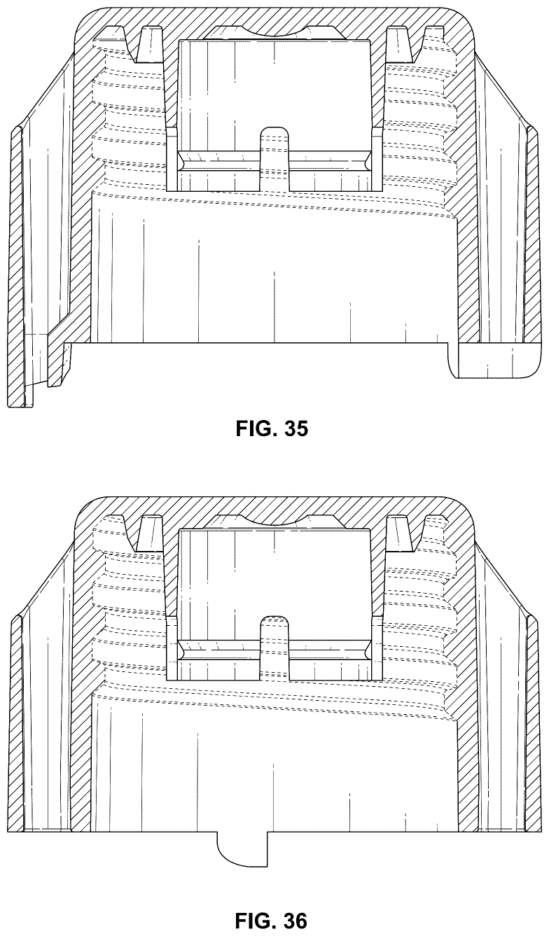

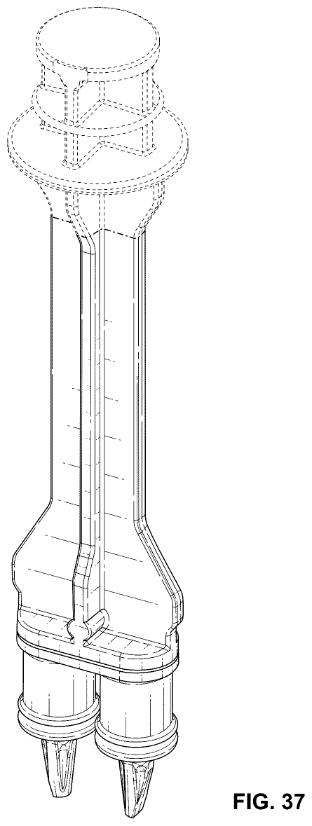

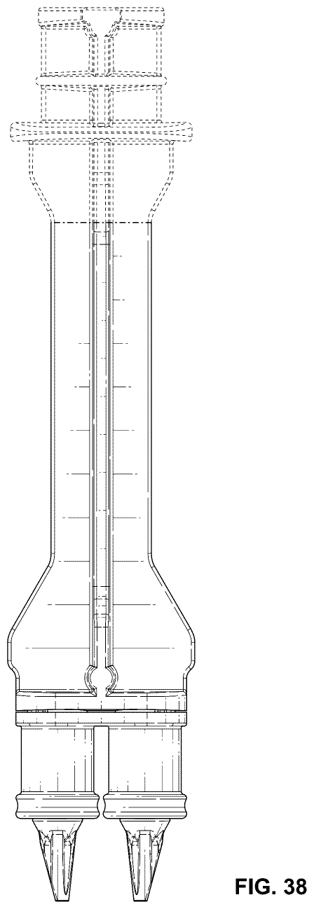

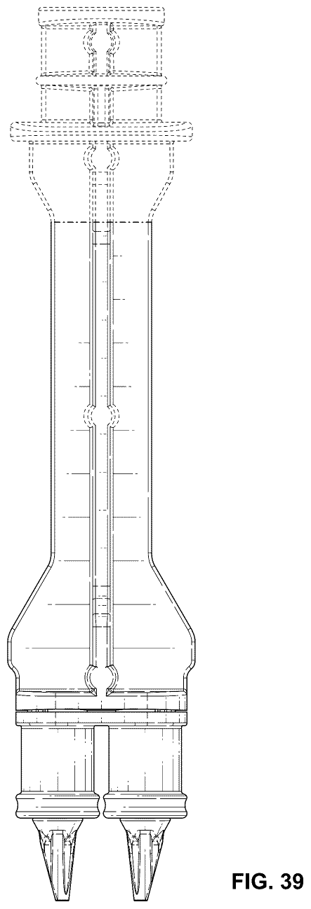

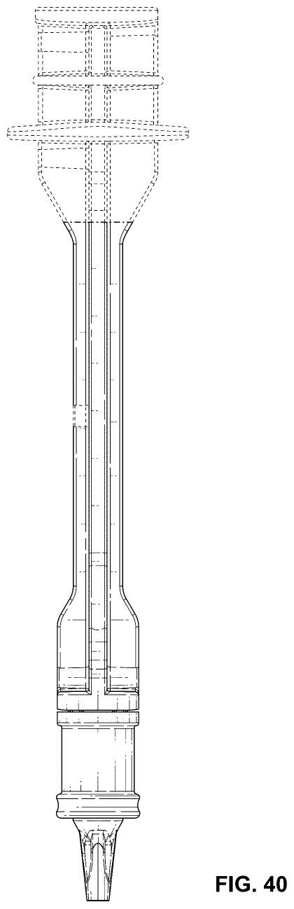

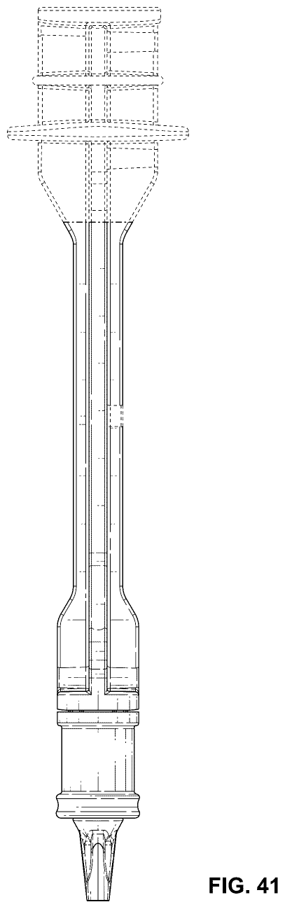

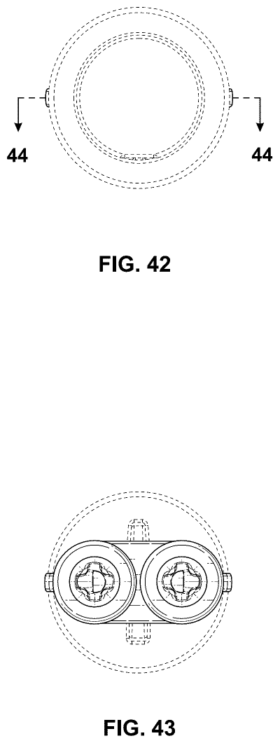

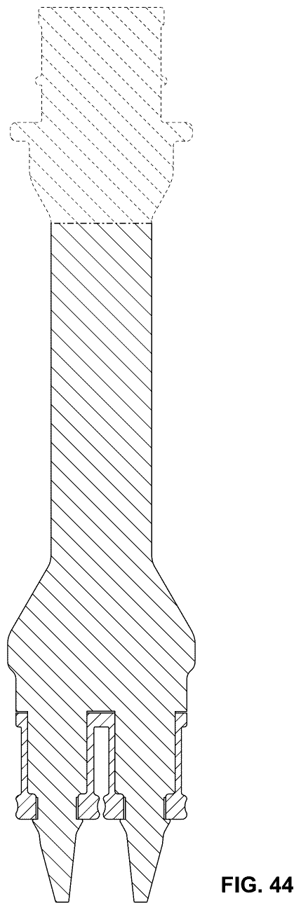

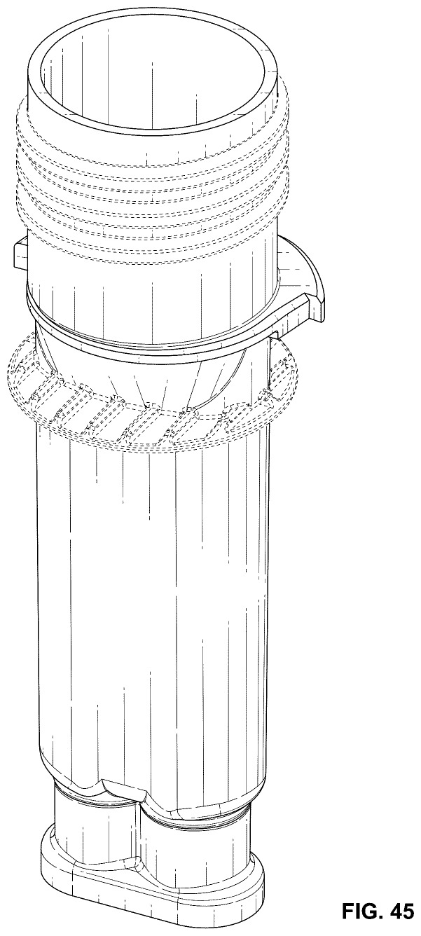

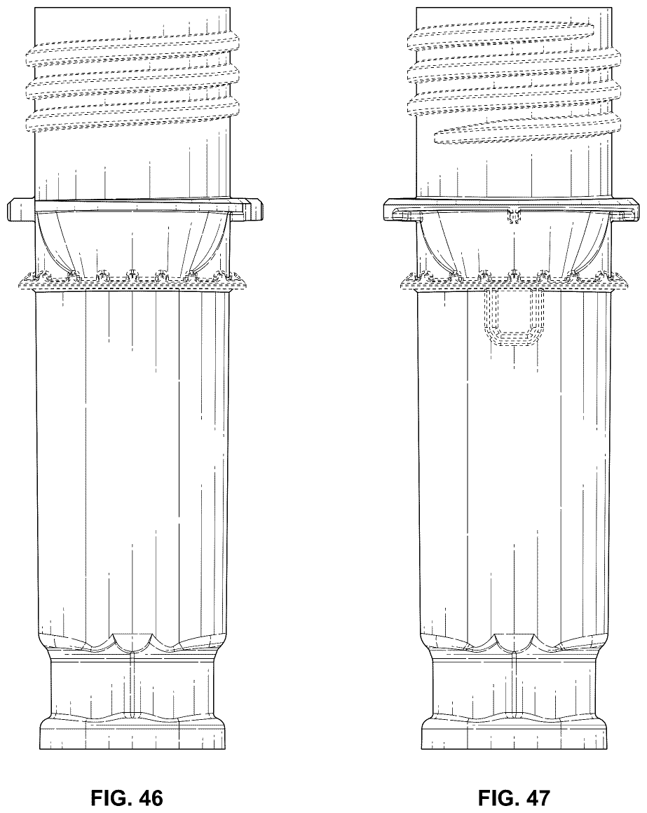

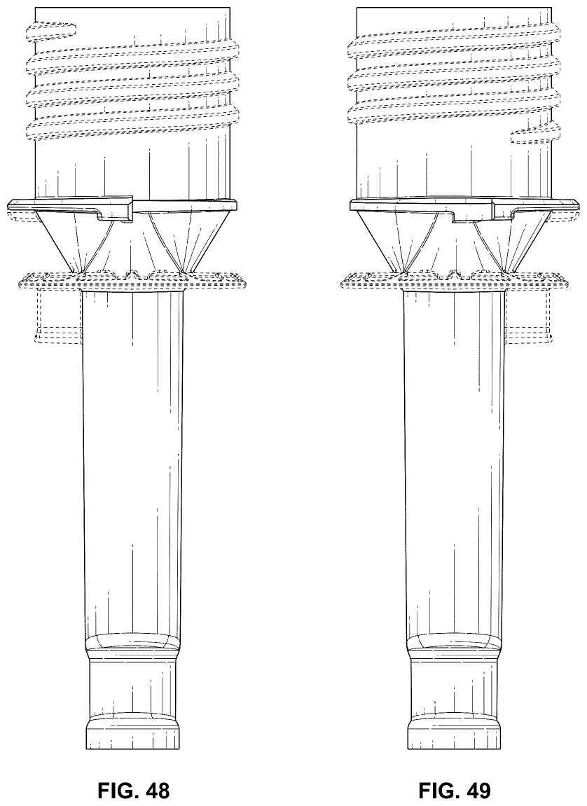

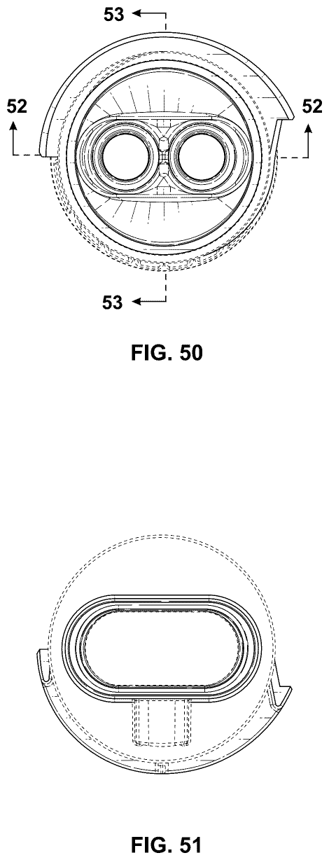

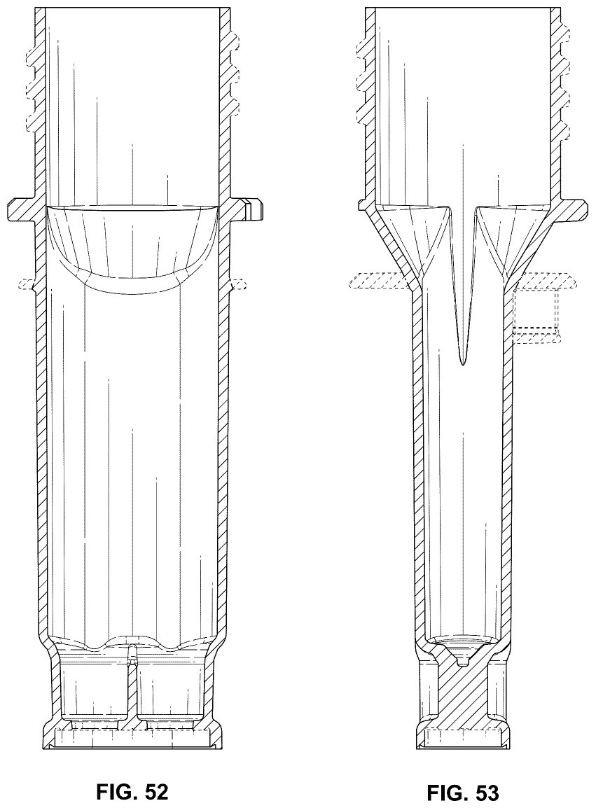

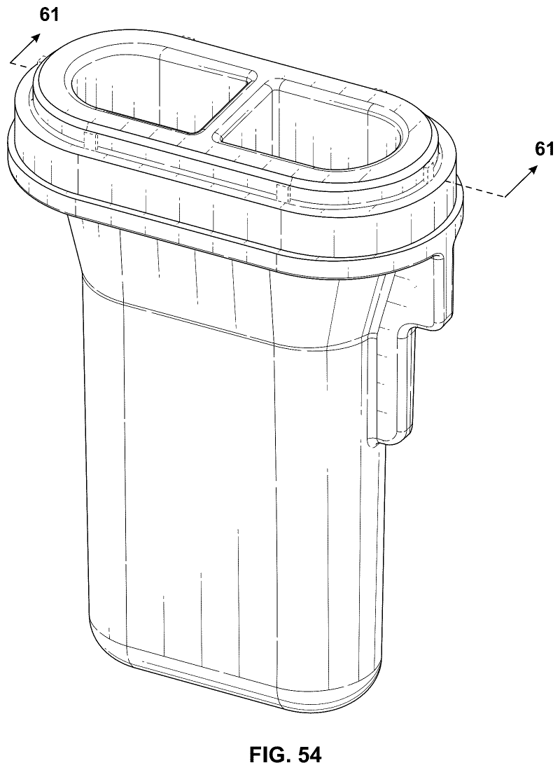

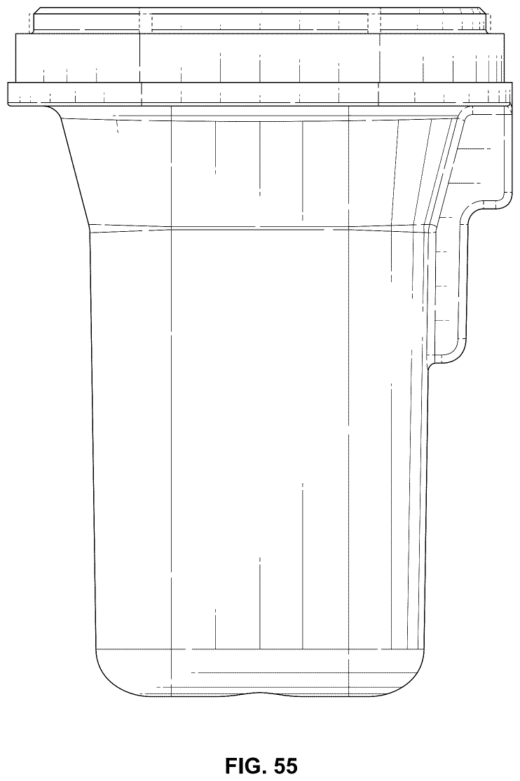

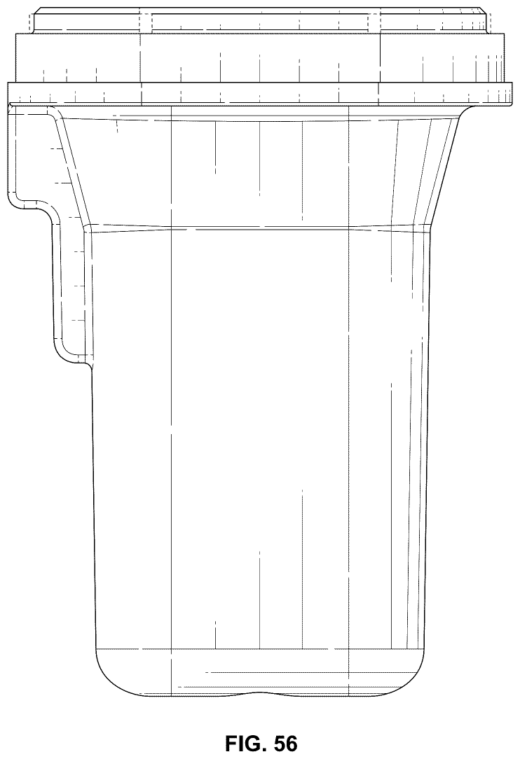

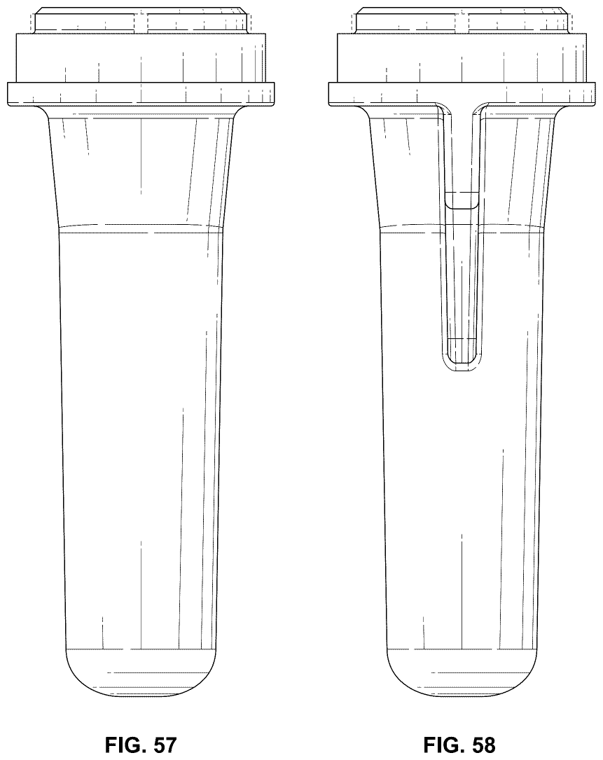

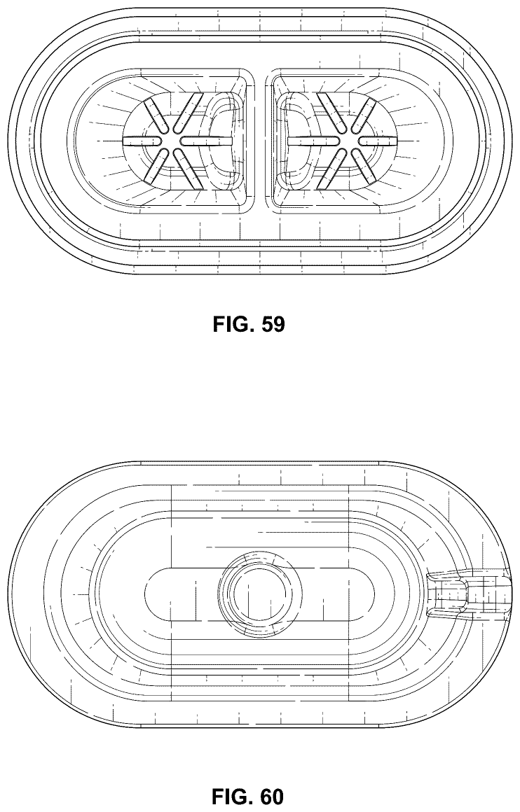

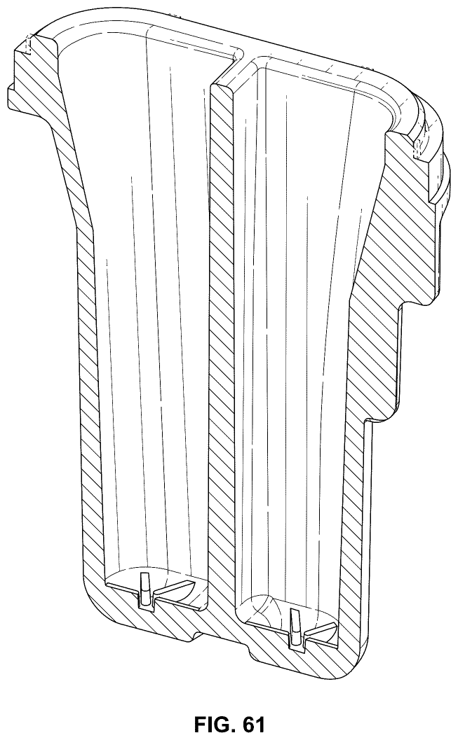

FIGS. 10-61 illustrate another non-limiting implementation of a diagnostic test device according to the present disclosure. FIGS. 10-18 illustrate perspective, front, rear, left, right, top, bottom, top exploded, and bottom exploded views, respectively, of the diagnostic test device including a dispense cap. FIGS. 19-27 illustrate perspective, front, rear, left, right, top, bottom, top exploded, and bottom exploded views, respectively, of the diagnostic test device including a transportation cap. FIGS. 28-34 illustrate perspective, front, rear, left, right, top, and bottom views, respectively, of the dispense cap of the diagnostic test device. FIG. 35 is a cross-sectional view taken along line 35-35 of FIG. 33. FIG. 36 is a cross-sectional view taken along line 36-36 of FIG. 33. FIGS. 37-43 illustrate perspective, front, rear, left, right, top, and bottom views, respectively, of the piercing member of the diagnostic test device. FIG. 44 is a cross-sectional view taken along line 44-44 of FIG. 42. FIGS. 45-51 illustrate perspective, front, rear, left, right, top, and bottom views, respectively, of the cartridge body of the diagnostic test device. FIG. 52 is a cross-sectional view taken along line 52-52 of FIG. 50. FIG. 53 is a cross-sectional view taken along line 53-53 of FIG. 50. FIGS. 54-60 illustrate perspective, front, rear, left, right, top, and bottom views, respectively, of the test container of the diagnostic test device. FIG. 61 is a cross-sectional view taken along line 61-61 of FIG. 54.

### Manually Operated, Visually Read, Non-Instrumented Operation of a Diagnostic Test Device

In some applications, the diagnostic test device 100 can be used manually without an instrument. For example, in some embodiments, the diagnostic test device 100 is held in one hand, and the transportation cap 116 removed with the other hand, the sample is added, the dispensing mechanism 102 is inserted into the cartridge body 108, and the dispense cap 114 is fitted to cartridge body 108 and rotated closed. In some such embodiments, where the diagnostic test reservoir(s) 204 are visually transparent, the dispensing of fluid into the diagnostic test reservoir(s) 204 can be visually observed, and a color or turbidity change observed over time to provide a diagnostic test readout or display. This approach uses the advantages of operating with a fully sealed cartridge 106 once the sample is added and internally dispensing a measured volume of prepared sample fluid into the diagnostic test reservoir(s) 204 without the use of external fluid transfer steps.

Optionally, a stand may be provided to support the diagnostic test device 100 for the purpose of removing the transportation cap 116, adding the sample, inserting the dispensing mechanism 102, and fitting, closing, and locking the dispense cap 114 to the cartridge body 108.

Optionally, a heater block may be provided to provide temperature control of the sample preparation reservoir 202 and diagnostic test reservoir(s) 204 of the diagnostic test device 100, but the diagnostic test device 100 is manually withdrawn to observe the test result visible in one or more diagnostic test reservoir(s) 204. In some applications, the heater block may include a window making the diagnostic test reservoir(s) 204 visible. In such applications, the diagnostic test device 100 need not be withdrawn from the heating block to observe the test result.

### Terminology

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount.

The term “and/or” as used herein has its broadest least-limiting meaning which is the disclosure includes A alone, B alone, both A and B together, or A or B alternatively, but does not require both A and B or require one of A or one of B. As used herein, the phrase “at least one of”′ A, B, “and” C should be construed to mean a logical A or B or C, using a non-exclusive logical or.

Conditional language used herein, such as, among others, “can,” “could,” “might,” “may,” and the like, unless specifically stated otherwise, or otherwise understood within the context as used, is generally intended to convey that certain features, elements and/or steps are optional. Thus, such conditional language is not generally intended to imply that features, elements and/or steps are in any way required. The terms “comprising,” “including,” “having,” and the like are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Any methods disclosed herein need not be performed in the order recited. The methods disclosed herein include certain actions taken by a practitioner; however, they can also include any third-party instruction of those actions, either expressly or by implication.

Some or all of the methods and tasks described herein may be performed and fully automated by a computer system. A diagnostic test system according to the present disclosure can include a computer system that may, in some cases, include multiple distinct computers or computing devices (for example, physical servers, workstations, storage arrays, cloud computing resources, etc.) that communicate and interoperate over a network to perform the described functions. Each such computing device typically includes a processor (or multiple processors) that executes program instructions or modules stored in a memory or other non-transitory computer-readable storage medium or device (for example, solid state storage devices, disk drives, etc.). The various functions disclosed herein may be embodied in such program instructions, and/or may be implemented in application-specific circuitry (for example, ASICs or FPGAs) of the computer system. Where the computer system includes multiple computing devices, these devices may, but need not, be co-located. The results of the disclosed methods and tasks may be persistently stored by transforming physical storage devices, such as solid-state memory chips and/or magnetic disks, into a different state. The computer system may be a cloud-based computing system whose processing resources are shared by multiple distinct business entities or other users.

While the above detailed description has shown, described, and pointed out novel features, it can be understood that various omissions, substitutions, and changes in the form and details of the devices, systems, and methods can be made without departing from the spirit of the present disclosure. As can be recognized, certain portions of the description herein can be embodied within a form that does not provide all of the features and benefits set forth herein, as some features can be used or practiced separately from others. Consequently, it is not intended that the present disclosure be limited to the specific embodiments disclosed herein, but that it covers all modifications and alternatives coming within the true scope and spirit of the present disclosure.

1. A device comprising:
a sample preparation reservoir configured to receive a sample at a first end and comprising an interior surface defining sides of at least one chamber at a second end;
at least one diagnostic test reservoir;
at least one seal disposed between the sample preparation reservoir and the at least one diagnostic test reservoir; and
a dispensing mechanism configured to be inserted into the first end of the sample preparation reservoir and translated toward the second end of the sample preparation reservoir, the dispensing mechanism comprising a piercing member and a sealing member, the sealing member configured to engage the sides of the at least one chamber as the dispensing mechanism translates toward the second end of the sample preparation reservoir, a predetermined volume of fluid defined between the sealing member, the piercing member, the sides of the at least one chamber at the second end of the sample preparation reservoir, and the at least one seal when the sealing member engages the sides of the at least one chamber, the piercing member configured to pierce the at least one seal after the predetermined volume is defined, and the sealing member and the piercing member configured to dispense the defined volume from the sample preparation reservoir to the at least one diagnostic test reservoir after the seal is pierced.

2. The device of claim 1, wherein the sealing member is configured to directly contact the interior surface defining the sides of the at least one chamber as the dispensing mechanism translates toward the second end of the sample preparation reservoir.

3. The device of claim 1, wherein the piercing member does not move relative to the sealing member as the defined volume is dispensed to the at least one diagnostic test reservoir.

4. The device of claim 1, wherein the piercing member comprises at least one spiked rod and the sealing member comprises at least one gasket encircling the at least one spiked rod.

5. The device of claim 1, wherein a single action of translating the dispensing mechanism toward the second end of the sample preparation reservoir (a) defines the predetermined volume of fluid between the sealing member, the piercing member, the sides of the at least one chamber, and the at least one seal; (b) pierces the at least one seal; and (c) dispenses the defined volume into the at least one diagnostic test reservoir.

6. The device of claim 1, wherein the sample preparation reservoir, the at least one diagnostic test reservoir, and the at least one seal are connected to form a joined structure.

7. The device of claim 1, wherein the interior surface at the second end of the sample preparation reservoir defines at least one cylindrical chamber.

8. The device of claim 7, wherein the interior surface at the second end of the sample preparation reservoir defines two cylindrical chambers.

9. The device of claim 8, further comprising a notch in a portion of the interior surface between the two cylindrical chambers, and wherein the predetermined volume is defined, at least in part, by a depth of the notch.

10. The device of claim 1, wherein the sealing member is configured to be in direct contact with a lower interior surface of the at least one chamber when the defined volume has been dispensed from the sample preparation reservoir to the at least one diagnostic test reservoir.

11. The device of claim 1, wherein the interior surface at the second end of the sample preparation reservoir defines two cylindrical chambers, each of the two cylindrical chambers configured to dispense the predetermined volume of fluid, wherein the piercing member comprises two spiked rods, wherein the sealing member comprises a gasket encircling each of the two spiked rods, and wherein the device further comprises a test container comprising two diagnostic test reservoirs, each diagnostic test reservoir configured to receive the predetermined volume of fluid from one of the two cylindrical chambers.

12. The device of claim 1, wherein the interior surface at the second end of the sample preparation reservoir defines four cylindrical chambers, and wherein the device comprises four diagnostic test reservoirs.

13. The device of claim 1, wherein the sealing member comprises an elastomeric material.

14. The device of claim 1, wherein the piercing member comprises one or more spikes.

15. The device of claim 14, wherein each of the one or more spikes comprises a cross-shaped cross-section comprising concave surfaces and a chamfered surface.

16. The device of claim 1, wherein the sample preparation reservoir comprises a sample preparation fluid.

17. The device of claim 1, wherein the at least one seal comprises a first seal configured to seal the second end of the sample preparation reservoir and a second seal configured to seal the diagnostic test reservoir.

18. The device of claim 1, wherein the at least one seal comprises a foil.

19. The device of claim 1, wherein the sample preparation reservoir is configured to receive a swab comprising the sample.

20. The device of claim 1, wherein the second end of the sample preparation reservoir comprises a lip configured to be joined to the diagnostic test reservoir.

21. The device of claim 1, wherein the sample preparation reservoir is configured to contain a volume of fluid ranging from 1-3 mL and the predetermined volume ranges between 10 μL and 1 mL.

22. The device of claim 1, wherein the sample preparation reservoir is configured to contain a fluid volume that is 1 to 300 times greater than the predetermined volume.

23. The device of claim 1, wherein the sample preparation reservoir is configured to contain a fluid volume of 1-3 mL and the predetermined volume is about 100 μL.

24. The device of claim 1, wherein the dispensing mechanism comprises a cap configured to engage the first end of the sample preparation reservoir.

25. The device of claim 24, wherein the cap is configured to rotate relative to the piercing member.

26. The device of claim 24, wherein the first end of the sample preparation reservoir comprises threads configured to engage threads of the cap.

27. The device of claim 24, wherein the cap is configured to lock to the first end of the sample preparation reservoir, preventing substantial motion of the cap relative to sample preparation reservoir, and wherein the cap comprises a plug seal configured to engage a top end of the sample preparation reservoir, the plug seal configured to block fluid flow when engaged to the top end of the sample preparation reservoir.

28. A diagnostic test apparatus configured to receive the device of claim 1.

29-45. (canceled)
